(19) United States
(12) Patent Application Publication
(10) Pub. No.: US 2025/0375188 A1
(43) Pub. Date: Dec. 11, 2025

(54) ULTRASOUND IMAGING DEVICE AND DOPPLER ULTRASOUND IMAGING METHOD THEREOF

(71) Applicant: SHENZHEN MINDRAY BIO-MEDICAL ELECTRONICS CO., LTD., Shenzhen (CN)

(72) Inventors: Lanxi XIANG, Shenzhen (CN); Shuangshuang LI, Shenzhen (CN)

(73) Assignee: SHENZHEN MINDRAY BIO-MEDICAL ELECTRONICS CO., LTD., Shenzhen (CN)

(21) Appl. No.: 19/231,369

(22) Filed: Jun. 6, 2025

(30) Foreign Application Priority Data
Jun. 7, 2024 (CN) 202410740864.0
Oct. 29, 2024 (CN) 202411527827.8

(51) Int. Cl. A61B 8/00; A61B 8/06; A61B 8/14

(52) U.S. Cl. CPC A61B 8/5246; A61B 8/06; A61B 8/14; A61B 8/463; A61B 8/5207; A61B 8/5223; A61B 8/5269; A61B 8/54

(57) ABSTRACT

Disclosed are an ultrasound imaging device and a Doppler ultrasound imaging method thereof, including: performing ultrasound scanning by alternately scanning between a complete B-mode ultrasound image and a complete C-mode ultrasound image during multimodal ultrasound imaging. In this way, when scanning the C-mode ultrasound image, although multiple samplings of a region of interest are still acquired, B-mode ultrasound image scanning is not interleaved between samplings. This reduces the sampling period, thereby increasing the velocity scale. During C-mode scanning, non-focused ultrasound waves are transmitted, which cover a larger scanning area in a single transmission. This further reduces the sampling period and enhances the velocity scale.

### CROSS-REFERENCE TO RELATED APPLICATION

This application claims priorities to and the benefits of Chinese Patent Application No. 202410740864.0, filed on Jun. 7, 2024 and Chinese Patent Application No. 202411527827.8, filed on Oct. 29, 2024, the disclosures of which are incorporated herein by reference in their entireties.

### TECHNICAL FIELD

The present disclosure relates to the field of medical devices, in particular to ultrasound imaging devices and Doppler ultrasound imaging methods thereof.

### BACKGROUND

Microvascular imaging, due to its clinical focus on low-velocity blood flow, requires a lower velocity scale range compared to conventional flow imaging. The velocity scale for conventional flow imaging typically ranges from 5 cm/s to 100 cm/s, whereas microvascular imaging uses a range generally set below 5 cm/s.

Current ultrasound imaging devices on the market often fail to support velocity scales exceeding 10 cm/s. Such low velocity scale configurations present the following technical limitations:

1. The use of lower velocity scales in Doppler flow imaging increases the likelihood of aliasing artifacts. When aliasing occurs, accurate detection of blood flow direction and velocity becomes impossible.

As shown in FIG. 1, the left figure depicts a multimodal ultrasound image exhibiting aliasing artifacts which essentially arise when the blood flow velocity exceeds the velocity scale, leading to clinically unreliable velocity data. The right figure shows a normal multimodal ultrasound image that captures a hemodynamic velocity, providing a diagnostically valid measurement.

2. The use of lower velocity scales has poor compatibility with high-velocity flow, and inadequately accommodate scenarios requiring simultaneous detection of high- and low-velocity flows (e.g., adult cardiac and fetal cardiac imaging). That is, excessively low velocity scales cause significant overflow of high-velocity within cardiac chambers, obscuring observations of fine branch blood flow.

To mitigate invalid velocity data caused by aliasing, conventional Doppler microvascular imaging modes in ultrasound devices typically do not support the detection of velocity and direction data, but display only single-energy information (which avoids aliasing due to the absence of directional components in energy information). This restriction impedes clinicians' ability to comprehensively assess microvascular flow.

Consequently, the upper limit of velocity scales in existing microvascular imaging systems still needs to be improved.

### SUMMARY

Ultrasound imaging devices and Doppler ultrasound imaging methods thereof disclosed herein are configured to increase the upper velocity scales in microvascular imaging detection.

A Doppler ultrasound imaging method for an ultrasound imaging device provided in some embodiments may include:
acquiring a current velocity scale;
according to the current velocity scale, controlling an ultrasound probe to perform B-mode scanning on target tissue and C-mode scanning on a region of interest (ROI) within the target tissue, wherein said controlling comprises alternately performing scanning by: (i) scanning at least one frame of B-mode ultrasound image followed by scanning at least one frame of C-mode ultrasound image, or (ii) scanning at least one frame of C-mode ultrasound image followed by scanning at least one frame of B-mode ultrasound image; wherein non-focused ultrasound waves are transmitted during the C-mode scanning;
processing echoes of the non-focused ultrasound waves transmitted during the C-mode scanning to obtain a hemodynamic parameter of the blood vessel within the ROI, said the hemodynamic parameter comprising at least one of: blood flow direction information, blood flow energy information and blood flow velocity magnitude information, and generating a C-mode ultrasound image showing the hemodynamic parameter based on the hemodynamic parameter of the blood vessel within the ROI; processing echoes of ultrasound waves transmitted during the B-mode scanning to obtain a B-mode ultrasound image of the target tissue;
generating a multimodal ultrasound image based on the C-mode ultrasound image showing the hemodynamic parameter and the B-mode ultrasound image of the target tissue, and displaying the multimodal ultrasound image, wherein at least a blood vessel in the C-mode ultrasound image showing the hemodynamic parameter has a diameter less than or equal to 200 um.

A Doppler ultrasound imaging method for an ultrasound imaging device provided in some embodiments may include:
controlling an ultrasound probe to perform B-mode scanning on target tissue and C-mode scanning on a ROI within the target tissue;
processing echoes of ultrasound waves transmitted during the C-mode scanning to obtain a hemodynamic parameter of a blood vessel within the ROI, said hemodynamic parameter comprising at least two of: blood flow direction information, blood flow energy information, and blood flow velocity magnitude information, generating a C-mode ultrasound image showing the hemodynamic parameter based on the hemodynamic parameter of the blood vessel within the ROI; wherein the C-mode ultrasound image comprises at least two of: a first C-mode ultrasound image, a second C-mode ultrasound image, and a third C-mode ultrasound image; the first C-mode ultrasound image is generated based on the blood flow energy information of the blood vessel in the ROI and is configured to show the blood flow energy information; the second C-mode ultrasound image is generated based on the blood flow direction information and the blood flow velocity magnitude information of the blood vessel in the ROI and is configured to show the blood flow direction information and the blood flow velocity magnitude information; the third C-mode ultrasound image is generated based on the blood flow direction information and the blood flow energy information of the blood vessel in the ROI and is configured to show the blood flow direction information and the blood flow energy information;
processing echoes of ultrasound waves transmitted during the B-mode scanning to obtain a B-mode ultrasound image of the target tissue;
generating a multimodal ultrasound image based on the B-mode ultrasound image and the C-mode ultrasound image and displaying the multimodal ultrasound image in real time;
wherein the generated multimodal ultrasound image comprises at least two of: a first multimodal ultrasound image, a second multimodal ultrasound image, and a third multimodal ultrasound image;
wherein the first multimodal ultrasound image is generated based on the B-mode ultrasound image and the first C-mode ultrasound image; the second multimodal ultrasound image is generated based on the B-mode ultrasound image and the second C-mode ultrasound image; and the third multimodal ultrasound image is generated based on the B-mode ultrasound image and the third C-mode ultrasound image.

A Doppler ultrasound imaging method for an ultrasound imaging device provided in some embodiments may include:
acquiring a current velocity scale;
based on the current velocity scale, controlling an ultrasound probe to perform B-mode scanning on target tissue and C-mode scanning on a ROI within the target tissue;
processing echoes of ultrasound waves transmitted during the C-mode scanning to obtain blood flow energy information of a blood vessel in the ROI, and, according to the blood flow energy information of the blood vessel in the ROI, generating a first C-mode ultrasound image showing the blood flow energy information; processing echoes of ultrasound waves transmitted during the B-mode scanning to obtain a B-mode ultrasound image;
based on the B-mode ultrasound image and the first C-mode ultrasound image, generating a first multimodal ultrasound image and displaying it in real time;
increasing the current velocity scale;
based on the increased velocity scale, controlling the ultrasound probe to perform B-mode scanning on the target tissue and C-mode scanning on the ROI within the target tissue, wherein said controlling comprises alternately performing scanning by: (i) scanning at least one frame of B-mode ultrasound image followed by scanning at least one frame of C-mode ultrasound image, or (ii) scanning at least one frame of C-mode ultrasound image followed by scanning at least one frame of B-mode ultrasound image, wherein non-focused ultrasound waves are transmitted during the C-mode scanning;
processing echoes of the non-focused ultrasound waves transmitted during the C-mode scanning to obtain blood flow direction information and blood flow velocity magnitude information of the blood vessel in the ROI, and based on the blood flow direction information and the blood flow velocity magnitude information of the blood vessel in the ROI, generating a second C-mode ultrasound image showing the blood flow direction information and the blood flow velocity magnitude information; processing echoes of ultrasound waves transmitted during the B-mode scanning to obtain a B-mode ultrasound image of the target tissue;
based on the B-mode ultrasound image and the second C-mode ultrasound image, generating a second multimodal ultrasound image, and displaying it in real time.

A Doppler ultrasound imaging method for an ultrasound imaging device is provided in some embodiments, wherein the ultrasound imaging device comprises two operational modes: (i) a Doppler microvascular imaging mode, preconfigured with a first velocity scale, and (ii) a Doppler conventional flow imaging mode, preconfigured with a second velocity scale, and the first velocity scale is lower than the second velocity scale; said method comprises:
when a current operational mode is the Doppler conventional flow imaging mode, setting the preconfigured second velocity scale of the Doppler conventional flow imaging mode as the current velocity scale;
based on the current velocity scale, controlling an ultrasound probe to perform B-mode scanning on target tissue and C-mode scanning on a ROI within the target tissue, wherein said controlling comprises: interleaving B-mode ultrasound image scanning during scanning of each frame of C-mode ultrasound image, wherein focused waves are transmitted during the C-mode scanning;
processing echoes of the focused waves transmitted during the C-mode scanning to obtain a hemodynamic parameter of the blood vessel within the ROI, said hemodynamic parameter comprising at least two of: blood flow direction information, blood flow energy information, and blood flow velocity magnitude information, and according to the hemodynamic parameter of the blood vessel within the ROI, generating a C-mode ultrasound image showing the hemodynamic parameter; processing echoes of ultrasound waves transmitted during the B-mode scanning to obtain a B-mode ultrasound image of the target tissue;
based on the C-mode ultrasound image showing the hemodynamic parameter and the B-mode ultrasound image of the target tissue, generating a multimodal ultrasound image and displaying it in real time;
in response to an instruction to switch the operational mode to the Doppler microvascular imaging mode, switching the operational mode to the Doppler microvascular imaging mode; setting the preconfigured first velocity scale of the Doppler microvascular imaging mode as the current velocity scale;
based on the current velocity scale, controlling the ultrasound probe to perform B-mode scanning on the target tissue and C-mode scanning on a ROI within the target tissue, wherein said controlling comprises alternately performing scanning by: (i) scanning at least one frame of B-mode ultrasound image followed by scanning at least one frame of C-mode ultrasound image, or (ii) scanning at least one frame of C-mode ultrasound image followed by scanning at least one frame of B-mode ultrasound image, and the ultrasound waves transmitted during the C-mode scanning are non-focused ultrasound waves;
processing echoes of the non-focused ultrasound waves transmitted during the C-mode scanning to obtain a hemodynamic parameter of the blood vessel within the ROI, said hemodynamic parameter comprises at least one of: blood flow direction information, blood flow energy information, and blood flow velocity magnitude information, and according to the hemodynamic parameter of the blood vessel within the ROI, generating a C-mode ultrasound image showing the hemodynamic parameter; processing echoes of ultrasound waves transmitted during the B-mode scanning to obtain a B-mode ultrasound image of the target tissue;
based on the C-mode ultrasound image showing the hemodynamic parameter and the B-mode ultrasound image of the target tissue, generating a multimodal ultrasound image, and displaying it in real time.

An ultrasound imaging device provided in some embodiments may include:
an ultrasound probe;
a transmit circuit, configured to excite the ultrasound probe to transmit ultrasound waves;
a receive circuit, configured to control the ultrasound probe to receive echoes of the ultrasound waves;
a processor, configured to execute the method mentioned above.

A computer-readable storage medium is provided in some embodiments, comprising a program stored thereon, wherein the program is executable by a processor to implement the method mentioned above.

The ultrasound imaging device and its Doppler ultrasound imaging method according to the above embodiments perform ultrasound scanning by alternately acquiring a complete B-mode ultrasound image and a C-mode ultrasound image during multimodal ultrasound imaging. During C-mode scanning, while multiple samplings of the ROI remain necessary, it eliminates the requirement for interleaved B-mode image acquisition between successive sampling sequences. This configuration reduces the sampling period, thereby increasing the velocity scale. Furthermore, the non-focused ultrasound waves transmitted during the C-mode scanning can cover a larger scanning region in a single transmission, which similarly reduces the sampling period and enhances the velocity scale.

### BRIEF DESCRIPTION OF THE DRAWINGS

FIG. 1 illustrates a comparison between an aliased multimodal ultrasound image and a normal multimodal ultrasound image;

FIG. 2 is a schematic diagram of conventional B-mode and C-mode interleaved scanning ultrasound beams in existing microvascular flow scenarios;

FIG. 3 is a schematic diagram of traditional focused wave imaging;

FIG. 4 is a schematically structural block diagram of an ultrasound imaging device in some embodiments;

FIG. 5 is a schematic flowchart a Doppler ultrasound imaging method in some embodiments according to the present disclosure;

FIG. 6 is a schematic diagram of sequential B-mode and C-mode scanning ultrasound beams;

FIG. 7 is a schematic diagram of data acquisition dimensions for C-mode ultrasound imaging;

FIG. 8 is a schematic diagram of plane wave imaging;

FIG. 9 is a schematic diagram of scanning mechanisms for tissue imaging and blood flow imaging in an ultrasound imaging device in some embodiments according to the present disclosure;

FIG. 10 is a schematic diagram of scanning mechanisms for tissue imaging and blood flow imaging in an ultrasound imaging device in other embodiments according to the present disclosure;

FIG. 11 is a schematic diagram of a first multimodal ultrasound image generated by an ultrasound imaging device in some embodiments according to the present disclosure;

FIG. 12 is a schematic diagram of a second multimodal ultrasound image generated by an ultrasound imaging device in some embodiments according to the present disclosure;

FIG. 13 is a schematic diagram of a third multimodal ultrasound image generated by an ultrasound imaging device in some embodiments according to the present disclosure;

FIG. 14 is a schematic flowchart a Doppler ultrasound imaging method in other embodiments according to the present disclosure;

FIG. 15 is a schematic flowchart a Doppler ultrasound imaging method in yet other embodiments according to the present disclosure;

FIG. 16 is a schematic diagram of a conventional sample volume configuration;

FIG. 17 is a schematic flowchart of a spectral Doppler measurement method in some embodiments;

FIG. 18 is a schematic flowchart of a spectral Doppler measurement method in other embodiments;

FIG. 19 is a schematic diagram comparing a conventional sample volume configuration and a sample volume configuration of the present disclosure; and

FIG. 20 is a schematically detailed flowchart of a Doppler ultrasound imaging method in some embodiments according to the present disclosure.

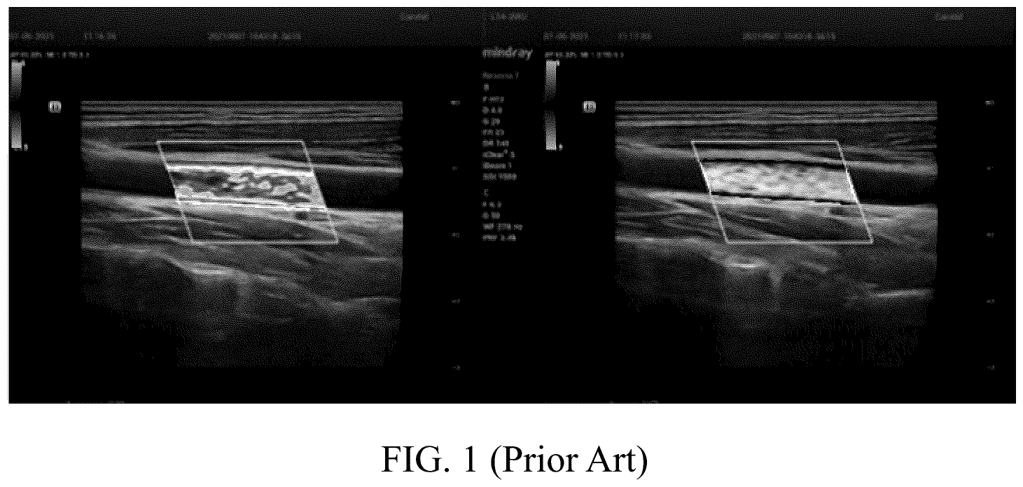

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusionis intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

Regarding the technical issues mentioned in existing technologies, through theoretical analysis and repeated experimentation, the inventors have discovered that the current technology exhibits a low velocity scale in microvascular flow imaging scenarios, primarily due to its scanning strategies in two aspects:
1. Interleaved scanning between B-mode and C-mode;
2. Utilization of focused wave scanning for C-mode.

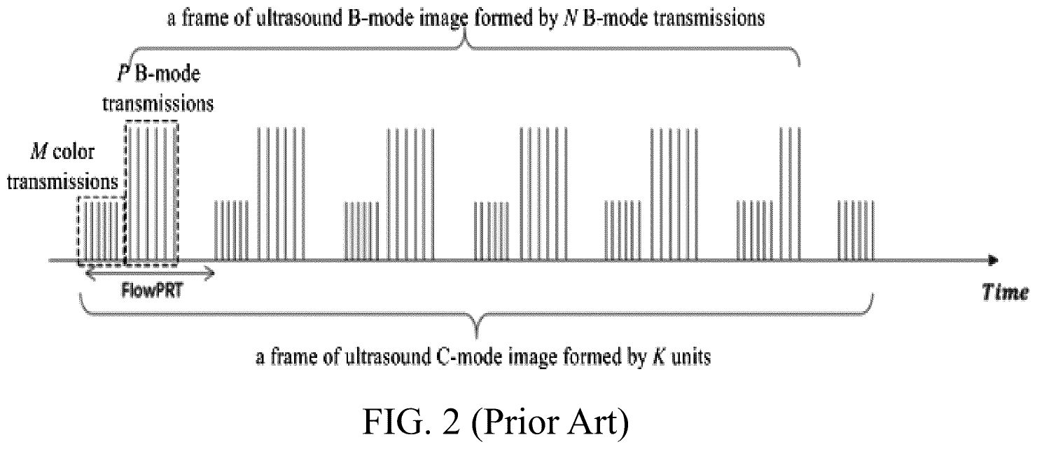

FIG. 2 illustrates an ultrasound scanning sequence in existing microvascular flow imaging, wherein the short-line portions represent ultrasound waves emitted during scanning of an ultrasound C-mode image (which may also be described as those emitted during C-mode scanning, hereinafter referred to as a “color transmission”); and the long-line portions represent ultrasound waves emitted during scanning of an ultrasound B-mode image (which may also be described as those emitted during B-mode scanning, hereinafter referred to as a “B-mode transmission”). A group of color emissions (comprising M color transmissions represented by short-lines within the dashed box) is referred to as a “unit”. Typically, each unit represents ultrasound emissions required for a single C-mode image sampling of a ROI (ROI). The temporal period of each unit is defined as FlowPRT. Between adjacent color units, B-mode image scanning is interleaved. The interleaved B-mode image scanning consists of a group of B-mode emissions (comprising P B-mode transmissions represented by long-lines within the dash box), which corresponds to a portion of a B-mode image. A complete frame of a B-mode image is reconstructed from a total of N such interleaved B-mode transmissions accumulated through multiple interleaved scans. Consequently, multiple alternating groups of color units and B-mode scanning sequences are required to ultimately acquire a frame of B-mode image and a frame of C-mode image.

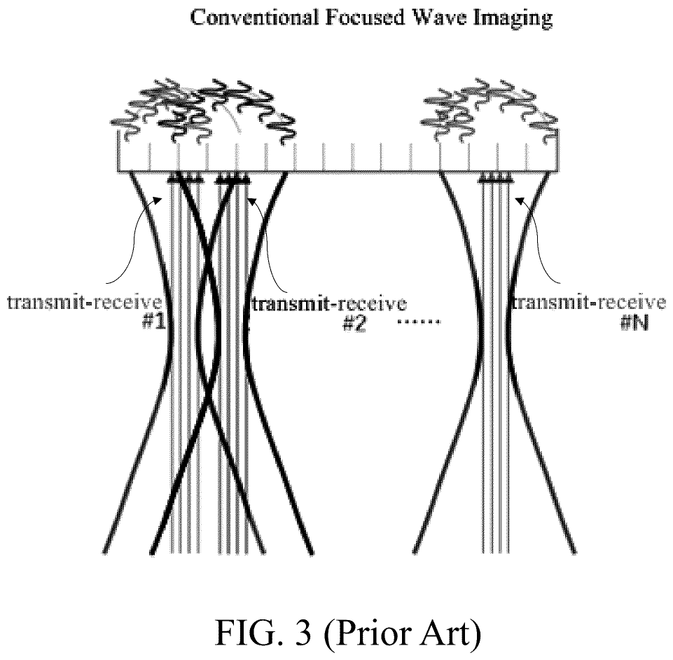

As shown in FIG. 3, since C-mode ultrasound imaging employs focused waves for scanning, spatial traversal of the ROI can be achieved by sequentially emitting the focused waves. The wider the ROI corresponding to the C-mode ultrasound image, the more emissions are required, which means a larger Min FIG. 2. When the ROI is large, FlowPRT tends to be prolonged, resulting in a lower velocity scale at the same imaging frequency. The quantitative relationship between the velocity scale and FlowPRT is defined as:

$$FlowPRT = \frac{{C}_{0}}{4 * {f}_{0} * {V}_{max}} ,$$

where: Vₘₐₓ represents the currently supported maximum measurement velocity (i.e., the velocity scale), f₀ represents the center frequency of the ultrasound waves used for imaging, and c₀ is the propagation speed of ultrasound waves in human tissue (which may be, for example, 1.54 mm/us). When using units of mm/us for c₀, MHz for f₀, and mm/s for Vₘₐₓ, FlowPRT is calculated in seconds.

Due to the adoption of the interleaved B-mode and C-mode scanning, B-mode scanning is mandatorily interleaved between adjacent color units, requiring a minimum of P B-mode transmissions to maintain acceptable B-mode frame rates. This mandatory interleaving increases the temporal interval between color units, thereby prolonging FlowPRT and reducing the velocity scale.

Due to the above defects, current ultrasound systems for microvascular imaging are limited to velocity scales below 10 cm/s. To avoid aliasing-induced velocity measurement inaccuracies, compared to conventional flow imaging, these systems do not support the measurement and display of velocity and direction data, and can only display single-energy micro-blood flow (as energy information remains unaffected by aliasing).

The present disclosure addresses these limitations through improved ultrasound imaging devices and Doppler methods, enabling measurement and display of both velocity magnitude and direction in microvascular imaging scenarios. Specific embodiments are detailed below.

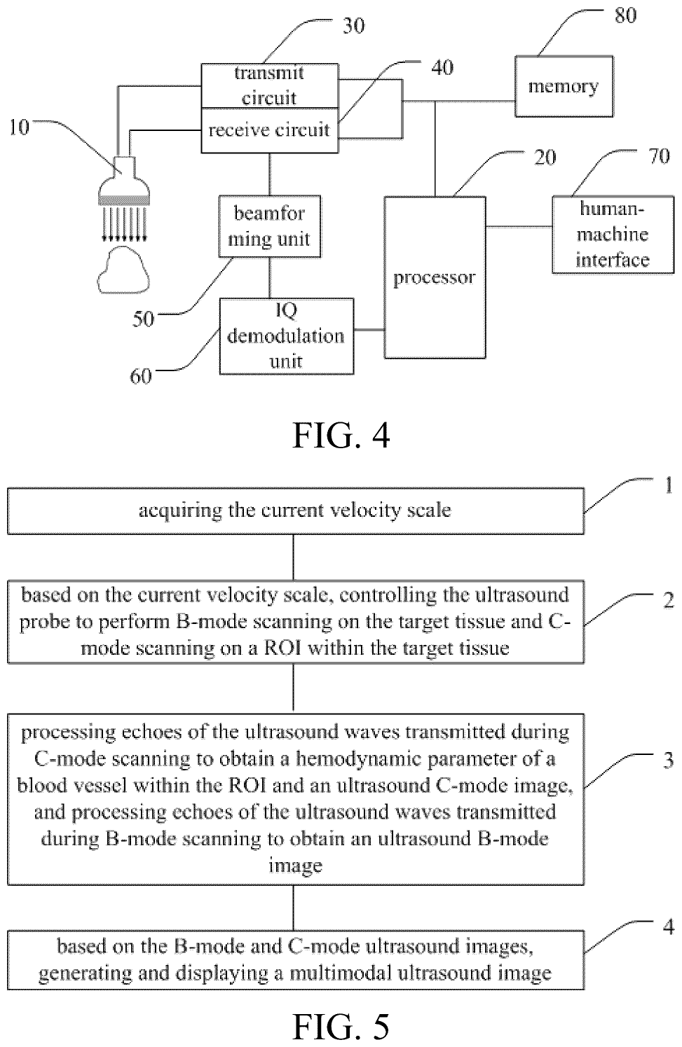

As shown in FIG. 4, an ultrasound imaging device disclosed herein may include an ultrasound probe 10, a transmit circuit 30, a receive circuit 40, a processor 20, and a memory 80.

The ultrasound probe 10 has a transducer comprising an array of multiple transducer elements (not shown in the figure). The transducer elements are configured to emit ultrasound waves in response to excitation electrical signals, or convert received ultrasound waves into electrical signals. Thus, each transducer element can achieve mutual conversion between electrical pulse signals and ultrasound waves, thereby: transmitting ultrasound waves toward biological tissues of a target object, and receiving echo waves of ultrasound waves reflected from the tissues.

The transmit circuit 30 is configured to excite the ultrasound probe 10 to transmit ultrasound waves. For example, under the control of the processor 20, it excites the ultrasound probe 10 to emit ultrasound waves toward a target object.

The receive circuit 40 is configured to control the ultrasound probe 10 to receive echoes of the ultrasound waves. For example, it can receive ultrasound echoes from the target object to obtain ultrasound echo signals. It may also process the ultrasound echo signals. The receive circuit 40 may include one or more amplifiers and analog-to-digital converters (ADCs).

The memory 80 is configured to store various types of data.

The ultrasound imaging device may further include a beamforming unit 50 and an IQ demodulation unit 60.

The beamforming unit 50 is signal-connected to the receive circuit 40 and configured to perform beamforming on the echo signals, including: applying time delays, and executing weighted summation. Since distances between ultrasound reception points in the tissue under examination (also referred to as the target tissue) and receiving transducer elements vary, channel data outputted from different receiving transducer elements corresponding to the same reception point exhibits time delay differences. Accordingly, the beamforming unit 50 can perform mandatory delay processing, align signal phases, execute weighted summation of multi-channel data from the same reception point, and obtain a beamformed ultrasound image data (referred to as RF data). The beamforming unit 50 can output the RF data to the IQ demodulation unit 60. In some embodiments, the beamforming unit 50 may alternatively output the RF data to the memory 80 for buffering/storage, or directly output the RF data to the processor 20 for image processing.

The beamforming unit 50 may implement the above functions through hardware, firmware, or software. It may be integrated within the processor 20 or implemented as a separate component. The present disclosure does not limit this configuration.

The IQ demodulation unit 60 is configured to remove signal carriers via IQ demodulation, extract tissue structure information contained in the signals, and perform noise reduction through filtering, thereby obtaining baseband signals (IQ data pairs). The IQ demodulation unit 60 is also configured to output the IQ data pairs to the processor 20 for image processing. In some embodiments, the IQ demodulation unit 60 may also output the IQ data pairs to the memory 80 for buffering/storage, enabling subsequent image processing by the processor 20 through the memory access.

The IQ demodulation unit 60 may implement the above functions through hardware, firmware, or software. It may be integrated within the processor 20 or implemented as a separate component. The present disclosure does not limit this configuration.

The processor 20 is configured as, but not limited to, the following electronic components capable of processing input data according to specific logical instructions: central controller circuitry (CPU), one or more microprocessors, graphics controller circuitry (GPU), or any other equivalent electronic components. The processor 20 is functionally operable to control peripheral electronic components by executing received commands or predetermined instructions, or perform data read and/or write operations on the memory 80, or process input data through execution of programs stored in the memory 80. Such processing operations include, but not limit to: executing one or more processing operations on acquired ultrasound data according to one or more operational modes, such as: adjusting or defining transmission parameters of ultrasound waves emitted by the ultrasound probe 10, generating various image frames for display, modifying content formats displayed on a display device, and regulating image display setting (e.g., ultrasound images, interface components, ROI positioning).

When receiving echo signals, the acquired ultrasound data may be processed by the processor 20 in real time during scanning, or temporarily stored in the memory 80 for near-real-time processing during online/offline operations.

The processor 20 may control operations of the transmit circuit 30 and the receive circuit 40, including alternating or concurrent activation of both the transmit circuit 30 and the receive circuit 40. The processor 20 may, based on user selection or program settings, determine appropriate operational modes (e.g., B-mode, C-mode, D-mode [Doppler mode]), generate transmission sequences corresponding to the current operational mode, and transmit the transmission sequences to the transmit circuit 30. Accordingly, the transmit circuit 30 controls the ultrasound probe 10 with appropriate transmission sequences to emit ultrasound waves.

The processor 20 may further be configured to process ultrasound data to generate a grayscale image representing signal intensity variations within the scanned region. The grayscale image, reflecting internal anatomical structures of tissues, are defined as a B-mode image. The processor 20 may output the B-mode image to the display device for visualization.

In some embodiments, the ultrasound imaging device may further include a human-machine interface 70. The human-machine interface 70 is configured to facilitate human-machine interactions, including outputting visual information and receiving user inputs. The human-machine interface 70 may include an input unit and at least a display device. The input unit is configured to receive user inputs through, but not limit to: keyboards, control buttons, mice, trackballs, touchpads, or touchscreens integrated with the display device. The display device is operable to show the aforementioned ultrasound images.

It is noted that the structural configuration shown in FIG. 4 is schematic and non-limiting. The device may include: more or fewer components than those illustrated in FIG. 4, or components arranged in different configurations relative to FIG. 4. All components shown in FIG. 4 may be implemented through hardware and/or software.

The processor 20 may control the ultrasound imaging device to perform vascular ultrasound examinations for Doppler ultrasound imaging. The specific process, as exemplified in FIG. 5, may comprise the following steps:

Step 1: the processor 20 acquires the current velocity scale. The velocity scale may be understood as the maximum measurable range of blood flow velocity. The current velocity scale may be a preconfigured velocity scale. For example, the processor 20 may acquire a predetermined velocity scale associated with the target tissue, and set the predetermined velocity scale associated with the target tissue as the current velocity scale, thus enabling different target tissues to be matched with appropriate velocity scales. Alternatively, the current velocity scale may be user-defined. For example, the processor 20 may receive a user-input velocity scale through the human-machine interface 70, and adopt the user-input velocity scale as the current velocity scale.

Step 2: the processor 20 can, based on the current velocity scale, control the ultrasound probe 10 to perform B-mode scanning on the target tissue and C-mode scanning on a ROI within the target tissue.

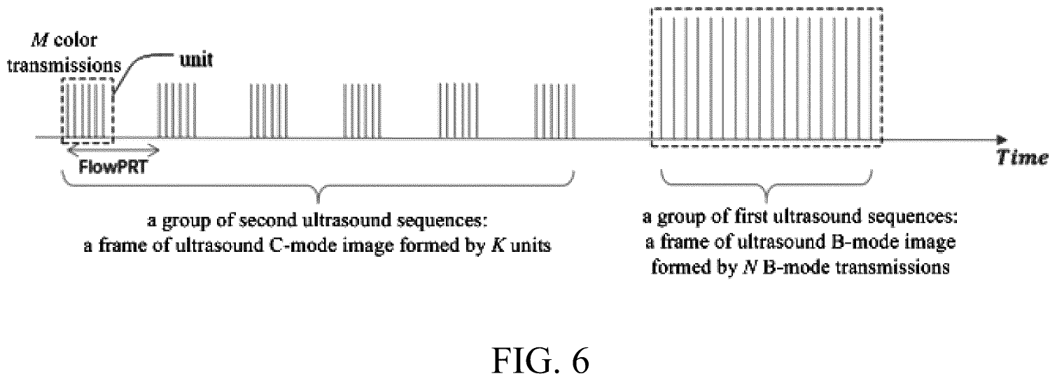

Unlike the interleaved scanning between B-mode and C-mode in conventional implementations of the prior art, the scanning method in the present embodiment employs sequential B-mode and C-mode scanning. The sequential B-mode and C-mode scanning involves alternately scanning B-mode ultrasound images and C-mode ultrasound images on a frame-by-frame basis. Specifically, one or more frames (typically one frame) of B-mode image are scanned followed by one or more frames (typically one frame) C-mode image, with this cycle repeated; or, one or more frames (typically one frame) of C-mode image are scanned followed by one or more frames (typically one frame) B-mode image, with this cycle repeated. For illustrative purposes, the present embodiment describes scanning one frame of B-mode or C-mode image followed by one frame of C-mode or B-mode image, i.e., alternately scanning by first obtaining a frame of B-mode ultrasound image followed by a frame of C-mode ultrasound image, or vice versa. To facilitate subsequent description, the ultrasound waves transmitted for scanning one frame of B-mode ultrasound image are defined as a group of first ultrasound sequences, and the ultrasound waves transmitted for scanning one frame of C-mode ultrasound image are defined as a group of second ultrasound sequences, wherein each group of first ultrasound sequences is configured to obtain one frame of tissue image for the target tissue, and each group of second ultrasound sequences is configured to obtain one frame of blood flow image for the ROI within the target tissue. That is, the processor 20 may, based on the current velocity scale, control the ultrasound probe 10 to alternately transmit the first and second ultrasound sequences on a group-by-group basis. This alternating transmission may follow either a group of first ultrasound sequences followed by a group of second ultrasound sequences, or a group of second ultrasound sequences followed by a group of first ultrasound sequences. Both alternating modes are functionally equivalent in operational principle. As shown in FIG. 6, the long-lines enclosed by dashed boxes represent a group of first ultrasound sequences, and the short-lines within K dashed boxed collectively represent a group of second ultrasound sequences. This configuration demonstrates that under the Doppler microvascular imaging mode, no B-mode ultrasound image scanning occurs in the intervals between successive samplings of the ROI. That is, during performing multiple samplings on the ROI to scan a frame of C-mode ultrasound image, B-mode ultrasound image scanning is entirely omitted.

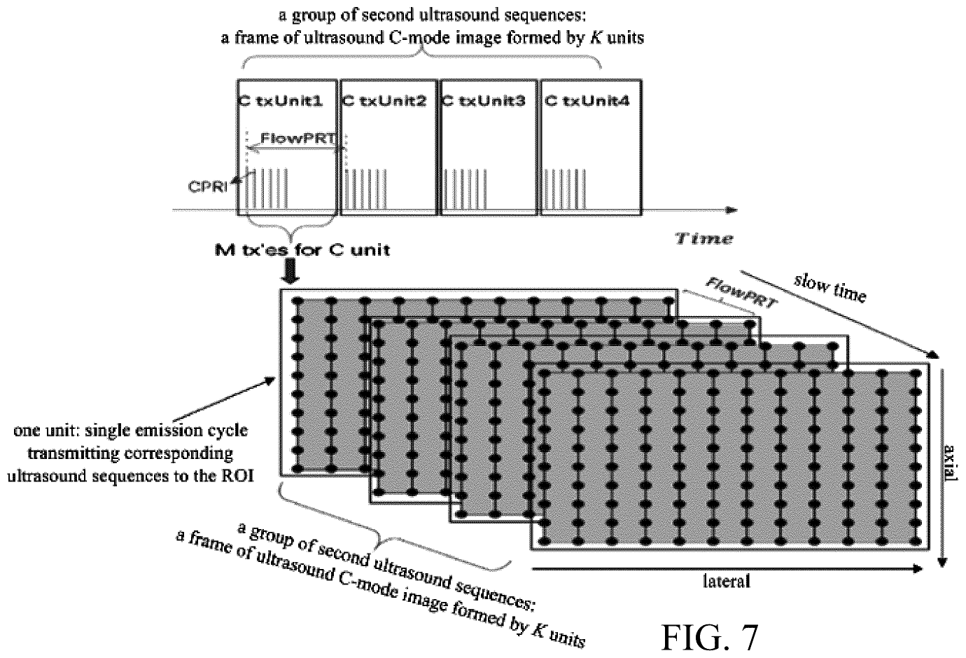

A group of second ultrasound sequences is schematically shown in FIG. 7. Doppler blood flow imaging is generally two-dimensional (2D) in spatial dimensions: lateral and axial. To extract Doppler signals, multiple samplings of blood flow signals at the same spatial location (e.g., the ROI) need to be performed at different time points (typically with fixed intervals). That is, scanning one frame of C-mode ultrasound image necessitates multiple samplings of the ROI. The corresponding sampling period, defined as FlowPRT, is inversely proportional to the velocity scale.

As shown in FIG. 7, the acquisition of Doppler raw data includes three dimensions: slow time (temporal dimension), lateral, and axial (spatial dimensions). Within a single acquisition unit, the C-mode scan achieves two-dimensional spatial traversal across the ROI. C-mode scans from different acquisition units correspond to sampling along the slow time dimension, meaning that generating one frame of C-mode ultrasound image requires multiple samplings of the ROI. The sampling interval in the slow time dimension is defined as the pulse-repetition time (PRT), also referred to herein as flow pulse-repetition time (FlowPRT). This parameter may alternatively be designated as the pulse repetition cycle (sampling period) or pulse repetition interval. FlowPRT is correlated with both the currently measured blood flow velocity range and the center frequency of the ultrasound signals. Specifically, when monitoring low-velocity blood flow (e.g., velocities below 5 cm/s), corresponding FlowPRT typically ranges between 1-10 ms. A narrower measured velocity range corresponds to a longer FlowPRT duration. Assuming N denotes the length of sampling sequences in the slow time dimension, the frame rate of blood flow imaging is calculated as: FrameRate=1/(N*FlowPRT). To ensure requisite temporal resolution in blood flow imaging, the frame rate must meet predefined operational thresholds.

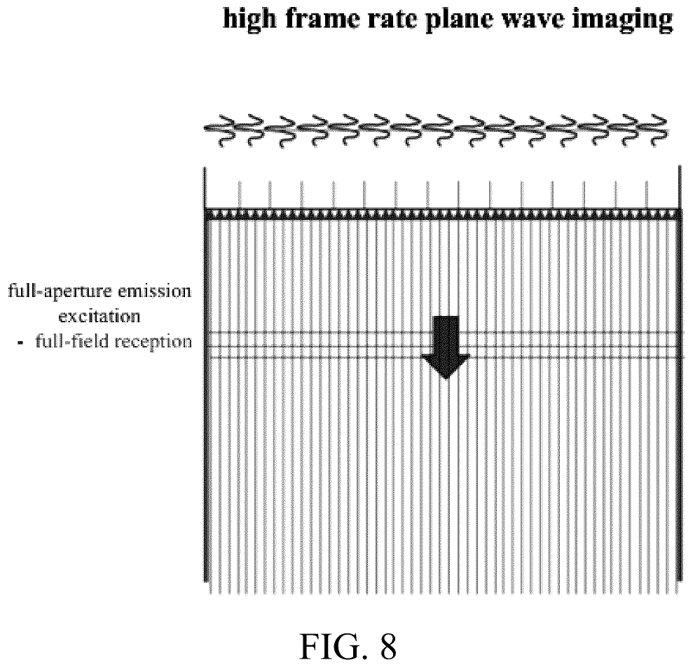

In conventional implementations of the prior art, focused waves are transmitted during the C-mode scanning, as shown in FIG. 3. When sampling with focused waves, each transmission can only capture echo information from a small region. To cover a certain two-dimensional spatial range (i.e., the ROI), multiple transmissions and receptions need to be performed sequentially from left to right. To enhance the velocity scale, the present embodiment employs non-focused ultrasound waves. The non-focused ultrasound waves may be plane waves or divergent waves, with FIG. 8 illustrating an example using plane waves. As shown in FIG. 8, a single transmission acquires echo information across a large region or even the entire field of view (ROI), resulting in a substantially smaller number of transmissions required compared to scanning with focused waves.

As shown in FIG. 7, let M be the number of ultrasound transmission within a single unit; then, FlowPRT≥M*CPRI, where CPRI represents a depth-dependent duration for a single C-mode scanning, which is the same for both the focused waves and plane waves. As can be seen from FIGS. 3 and 8, for the same size ROI: when using focused waves for transmission, M (typically above 30, increasing with the width of the ROI) is greater than that when using plane waves for transmission (typically 5-17, independent of the width of the ROI). This means that focused waves may require dozens of transmissions (M value) to fully cover the entire ROI, whereas plane waves may potentially cover the entire ROI with a single transmission, though emissions may be made from multiple different angles M times to optimize image quality, where M here is significantly smaller than that for focused waves. Therefore, when using plane waves for scanning, the value range of FlowPRT is larger than that for focused wave scanning, allowing for a smaller FlowPRT to be achieved. This means that using plane waves (and similarly divergent waves) can result in a higher velocity scale.

By comparing FIG. 6 (sequential B-mode and C-mode scanning) with FIG. 2 (interleaved scanning between B-mode and C-mode), it can be observed that under equal M values, the FlowPRT in FIG. 2 needs to satisfy FlowPRT>M*CPRI+P*BPRI, where CPRI represents the time required for one color transmission, and BPRI represents the time required for one B-mode transmission. In contrast, FIG. 6 only requires FlowPRT>M*CPRI. This demonstrates that the permissible range of FlowPRT values for the scanning mode shown in FIG. 6 is broader than that of the scanning mode shown in FIG. 2. That is, when employing the sequences of the sequential B-mode and C-mode scanning shown in FIG. 6, smaller FlowPRT values can be achieved, which corresponds to a larger velocity scale.

By integrating the two aforementioned enhancements in scanning strategies, namely, the use of non-focused ultrasound waves for color transmissions (C-mode scanning) and sequential B-mode and C-mode scanning, a broader velocity scale can be achieved compared to existing techniques (where focused waves are used for color transmissions and interleaved scanning between B-mode and C-mode are employed). Precisely because this approach supports a wider velocity scale, ultrasound imaging devices implementing the proposed method enhance their microvascular flow functionality, enabling the provision of richer hemodynamic information. In addition to energy information, they can also provide directional and velocity data of blood flow.

Step 3: the processor 20 processes echoes of the non-focused ultrasound waves transmitted during C-mode scanning to obtain a hemodynamic parameter of a blood vessel within the ROI. The hemodynamic parameter comprises at least one of blood flow direction information, blood flow energy information, and blood flow velocity magnitude information. The processor 20 generates a C-mode ultrasound image showing the hemodynamic parameter (a blood flow image) based on the hemodynamic parameter of the blood vessel within the ROI.

The processor 20 processes echoes of ultrasound waves transmitted during the B-mode scanning to obtain a B-mode ultrasound image of the target tissue. For example, the processor 20 processes echo signals of each group of first ultrasound sequences to obtain a frame of B-mode ultrasound image of the target tissue (a tissue image).

The processor 20 processes echoes of the ultrasound waves transmitted during C-mode scanning to obtain a hemodynamic parameter of the blood vessel within the ROI, and generates a C-mode ultrasound image showing the hemodynamic parameter based on the hemodynamic parameter of the blood vessel within the ROI. The implementation details may vary; and two examples are provided below.

First Implementation: The echo signals corresponding to the second ultrasound sequences (i.e., the echo data) contain both tissue signals (e.g., tissue motion signals) and blood flow signals (e.g., blood flow motion signals), and the signal intensity of tissue signals is significantly stronger than that of blood flow signals (e.g., the intensity of tissue signals is approximately 100 to 1000 times greater). To effectively extract blood flow signals from the echo data, wall filtering is required to be applied to the echo signals.

Conventional Doppler wall filtering typically employs a pure time-domain high-pass filter. This approach exploits the characteristic that tissue motion generally exhibits low-velocity behavior, filtering out low-frequency tissue components in the temporal frequency domain while retaining higher-frequency blood flow signals. To ensure temporal resolution in blood flow imaging (where the frame rate requirement is typically ≥20 frames), the length of the sampling sequences in the slow time dimension is usually limited to 4-16. Consequently, the filter order in the slow time dimension is constrained by this sequence length. To achieve better filtering performance under low-order conditions (e.g., minimizing transition band width), traditional Doppler blood flow systems commonly utilize 4th-16th order IIR high-pass filters for wall filtering.

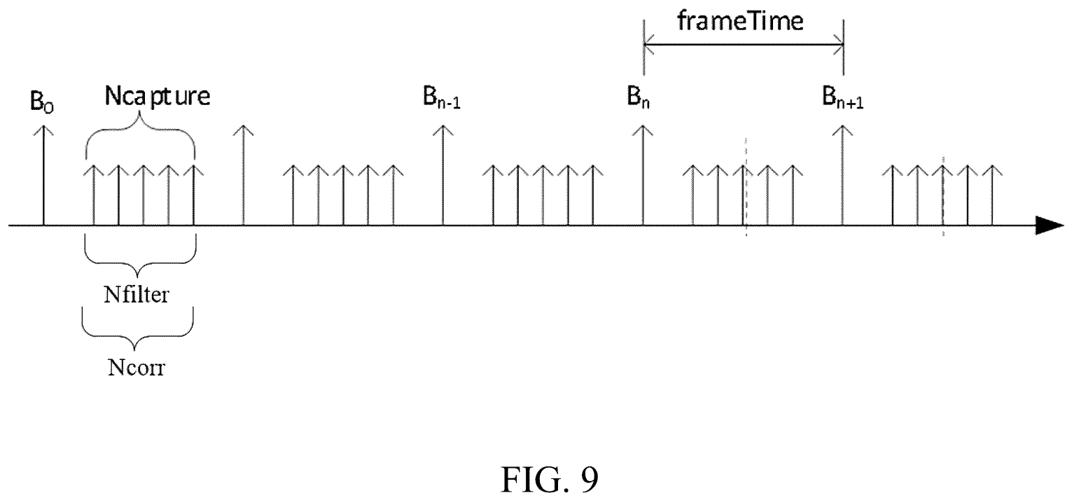

After wall filtering removes tissue components from the acquired echo signals, autocorrelation calculation may be employed to extract the hemodynamic parameter. Autocorrelation calculation is an operation performed in the slow time dimension, where the sequence length for this calculation is directly proportional to the signal-to-noise ratio of the blood flow signals. As shown in FIG. 9, in traditional Doppler blood flow systems, the autocorrelation sequence length Ncorr equals the filter order Nfilter used in wall filtering, which is also equivalent to the acquisition length Ncapture in the slow time dimension during echo signal collection (i.e., the length of one group of second ultrasound sequences). In FIG. 9, B₀-Bₙ₊₁ denote Groups 0 to n+1 of the of first ultrasound sequences.

In this method, the workflow of conventional Doppler blood flow imaging is adopted. Specifically, the processor 20 applies spatiotemporal wall filtering to the echo signals corresponding to a group of second ultrasound sequences to remove tissue motion signals, thereby obtaining blood flow motion signals. Autocorrelation calculation is then performed on these blood flow signals to obtain the hemodynamic parameters, which are used to generate a single frame of C-mode ultrasound image (a blood flow image). That is, the echo signals corresponding to one group of second ultrasound sequences produce one frame of C-mode ultrasound image. By repeating this process for echo signals of each group of second ultrasound sequences, a real-time C-mode ultrasound image is obtained. Here, the length of one group of second ultrasound sequences Ncapture equals the wall filter order Nfilter, which also matches the autocorrelation sequence length Ncorr.

This implementation is well-suited for the Doppler conventional flow imaging mode, which is typically used to examine blood vessels with larger diameter. In such vessels, higher blood flow velocities allow the use of lower blood flow detection sensitivity and spatial resolution in exchange for higher velocity scale. That is, for low-velocity blood flow, a challenge arises in the time-frequency domain: low-velocity blood flow signals overlap with low-velocity tissue motion signals; and pure time-domain filters may struggle to distinguish between the two. To suppress tissue motion interference, a significant portion of low-velocity blood flow signals may also be inadvertently filtered out. Consequently, accurate detection of low-velocity blood flow signals becomes difficult, resulting in reduced sensitivity in blood flow detection.

In clinical practice, superb microvascular imaging (SMI) prioritizes detecting smaller-diameter vessels with slower flow velocities, whereas conventional blood flow imaging focuses on medium-to-large vessels with relatively higher flow velocities (typically exceeding 5 cm/s). Consequently, SMI demands significantly higher blood flow detection sensitivity and spatial resolution compared to conventional blood flow imaging.

In this embodiment, the echo signals from the second ultrasound sequences are processed using the second implementation which enables precise detection of blood flow velocities and directions for SMI, thereby allowing the Doppler microvascular imaging mode to not only capture energy information but also measure blood flow velocity and direction.

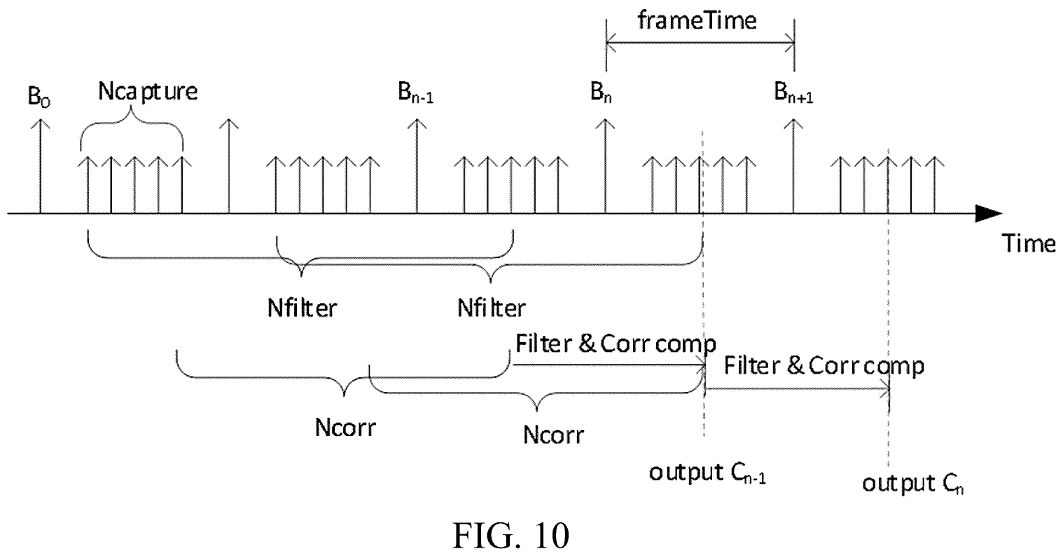

Second Implementation: The processor 20 processes echo signals corresponding to more than one group of second ultrasound sequences to generate a frame of C-mode ultrasound image, meanwhile, spatiotemporal wall filtering and autocorrelation calculations for each frame of C-mode ultrasound image utilize partially overlapped data segments across consecutive frames. As specially shown in FIG. 10, the processor 20 selects echo signals corresponding to the first sequence length Nfilter from the multiple groups of second ultrasound sequences as raw signals for spatiotemporal wall filtering, then performs spatiotemporal wall filtering on these raw signals to suppress tissue motion signals within the echo signals, thereby extracting blood flow motion signals. These blood flow motion signals include: a first part of blood flow motion signals with blood flow velocities within tissue motion velocity range, and a second part of blood flow motion signals with blood flow velocities exceeding the tissue motion velocity range. The processor 20 performs autocorrelation calculations on the blood flow motion signals to obtain the hemodynamic parameter, which is then used to generate a frame of C-mode ultrasound image. The autocorrelation calculations may be applied to a subset of the blood flow motion signals. For example, the processor 20 extracts a second sequence length Ncorr from the blood flow motion signals for autocorrelation analysis to obtain the hemodynamic parameter where the second sequence length Ncorr is greater than or equal to the length Ncapture of a group of second ultrasound sequences, and is smaller than or equal to the first sequence length Nfilter. Adjacent frames of C-mode ultrasound images exhibit overlapping segments in the echo signals corresponding to the first sequence length Nfilter, and may also exhibit overlapping in the blood flow motion signals corresponding to the second sequence length Ncorr. A larger Ncorr improves sensitivity for blood flow detection but may reduce the dynamic perception of blood flow. The specific value of Ncorr is determined by balancing sensitivity for blood flow detection and temporal resolution. In this embodiment, the second sequence length Ncorr is set to be greater than or equal to the length Ncapture of a group of second ultrasound sequences, and less than or equal to the first sequence length Nfilter. This configuration ensures sufficient sensitivity for blood flow detection detection while improving temporal resolution to maintain smooth dynamic rendering of the blood flow image. By expanding the data range for spatiotemporal wall filtering and autocorrelation calculations without altering the frame rage (i.e., the lengths of the first and second ultrasound sequences), this implementation enhances both sensitivity and temporal resolution on blood flow detection, enabling effective measurement of velocity and direction for SMI that conventional techniques fail to detect.

In FIG. 10, the lateral axis represents the slow time domain. The frame cycle (FrameTime) covers the scanning duration of one frame of Doppler blood flow (a multimodal ultrasound image), including B-mode scanning (long arrow) and blood flow scanning (short arrow). The sequence length of a frame of blood flow scanning in slow time is Ncapture (i.e., the length of each group of second ultrasound sequences is Ncapture). The sequence length of blood flow required by the spatiotemporal adaptive wall filter (i.e., the first sequence length) is Nfilter (Nfilter>Ncapture), and the final sequence length of the blood flow signals (referred to as the blood flow motion signals) used for autocorrelation calculations (i.e., the second sequence length) is Ncorr. Optionally, Ncapture≤Ncorr≤Nfilter and Ncapture<Nfilter. The final output blood flow image (C-mode image) is obtained by processing echo signals of length Nfilter through wall filtering to extract blood flow motion signals, then performing autocorrelation calculations on the sequence with the second sequence length Ncorr from the blood flow motion signals. As shown in FIG. 10, each frame of blood flow data acquisition strictly maintains the acquisition sequence length Ncapture (ensuring constant frame rate), while subsequent spatiotemporal wall filtering and autocorrelation processing apply sliding windows of lengths Nfilter and Ncorr, respectively. Each new frame of C-mode image is generated by sliding the window backward by Ncapture in original echo data. The blood flow image Cn and tissue image Bn are subsequently superimposed and paired in a one-to-one correspondence (forming a multimodal ultrasound image) for display on the interface of the display device.

Using the first implementation (conventional method), where the sequence length in the slow time dimension is significantly increased (e.g., to 60-200), it is theoretically possible to detect the velocity and direction of blood flow for SMI. However, this causes Ncapture to become excessively large, resulting in significantly reduced frame rates that fail to meet real-time Doppler blood flow imaging requirements. In contrast, the second implementation enhances the filtering efficacy of spatiotemporally adaptive wall filtering without altering the frame rate, thereby enabling real-time detection of blood flow velocities and directions for SMI. In one embodiment of the second implementation: the first sequence length Nfilter is set to greater than or equal to 60, and/or the first sequence length Nfilter exceeds four times the length of a single group of second ultrasound sequences. This configuration provides sufficient data for spatiotemporal wall filtering processing, meeting the stringent requirements for spatiotemporal consistency and high spatial resolution in SMI.

Step 4: the processor 20 generates a multimodal ultrasound image based on the C-mode ultrasound image showing the hemodynamic parameter and the B-mode ultrasound image of the target tissue, and displays the multimodal ultrasound image. The B-mode ultrasound image and the C-mode ultrasound image are obtained by B-mode and C-mode ultrasound scanning, respectively. Since the image formed by the two contains multidimensional information (e.g., tissue and blood flow), it is referred to as a multimodal ultrasound image or alternatively as a Doppler color image. Specifically, the processor 20 superimposes the B-mode ultrasound image onto the C-mode ultrasound image to obtain the multimodal ultrasound image. The C-mode ultrasound image covers only the ROI, so the C-mode ultrasound image is overlaid onto the corresponding ROI of the B-mode ultrasound image, thereby obtaining the multimodal ultrasound image. The multimodal ultrasound image is then displayed on the interface of the display device. That is, through the multimodal ultrasound image, users can simultaneously visualize the grayscale target tissue anatomy (B-mode image), as well as the blood vessel and its hemodynamic parameter (C-mode image), where color saturation represents blood flow velocity/energy and hue variations indicate flow direction. In the multimodal C-mode ultrasound image, at least one blood vessel has a diameter less than or equal to 200 um, demonstrating successful microvascular flow imaging and detection through the described method. The workflow shown in FIG. 5 operates in real time, enabling live display of the multimodal ultrasound image.

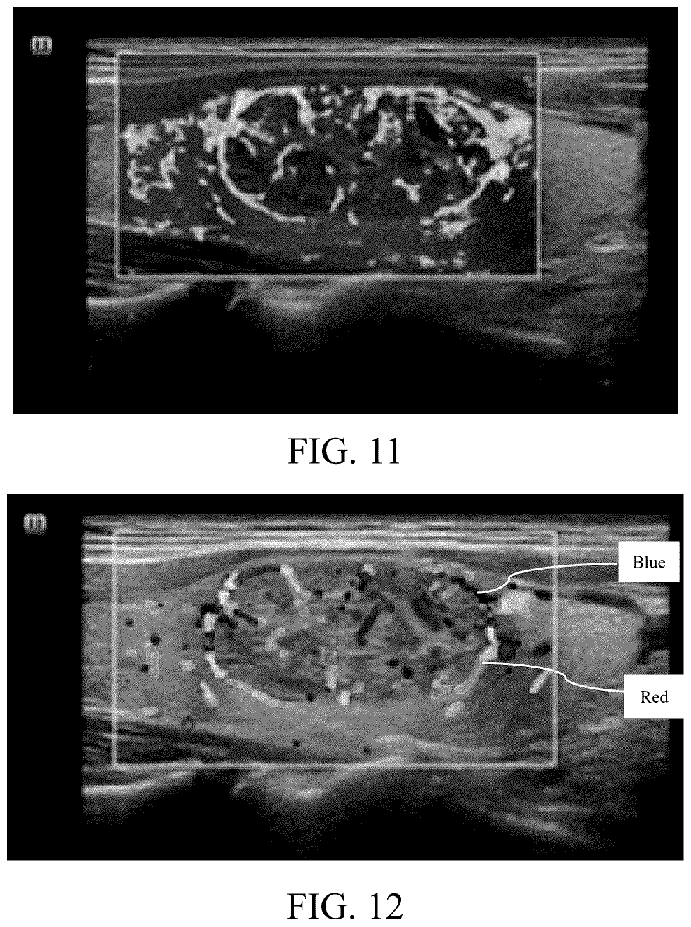

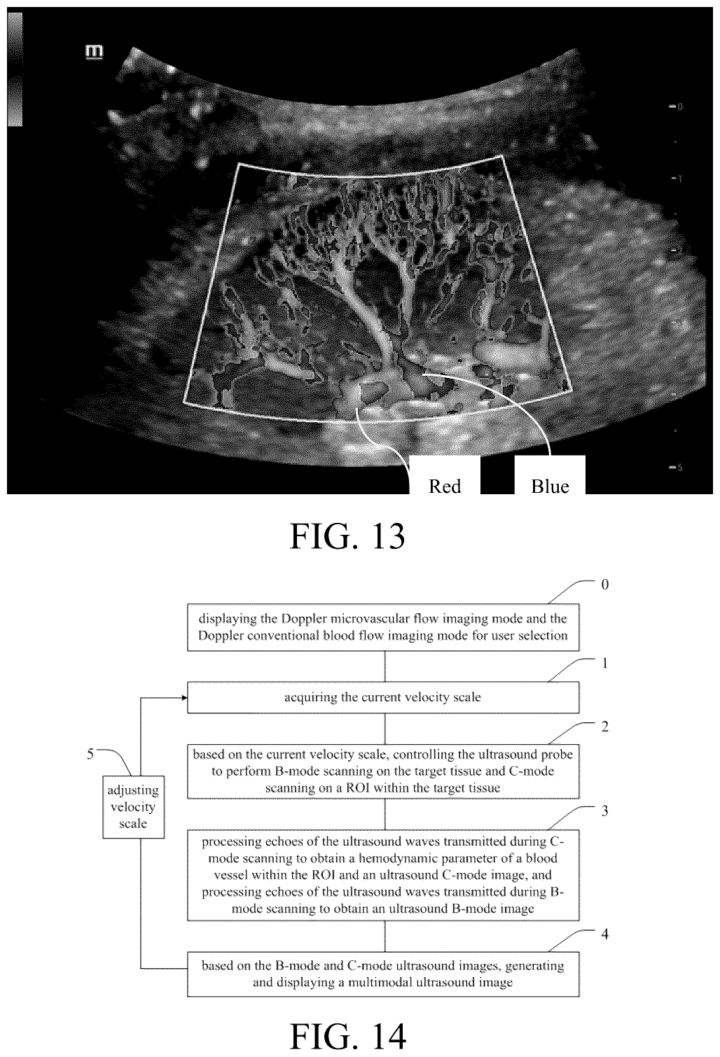

The Doppler ultrasound imaging method provided herein enables detection of blood vessel velocity, direction and energy in microvessels. Consequently, multiple types of C-mode ultrasound images can be generated in Step 3, and correspondingly, multiple types of multimodal ultrasound images can be produced in Step 4. For example, the C-mode ultrasound image generated in Step 3 include at least one of the following: a first C-mode ultrasound image, a second C-mode ultrasound image, and a third C-mode ultrasound image. Correspondingly, the multimodal ultrasound image generated in Step 4 include at least one of the following: a first multimodal ultrasound image, a second multimodal ultrasound image, and a third multimodal ultrasound image. The first C-mode ultrasound image is generated based on the blood flow energy information of the blood vessel in the ROI, and is configured to show the blood flow energy information; and correspondingly, in Step 4, the processor 20 can generate a first multimodal ultrasound image based on the B-mode ultrasound image and the first C-mode ultrasound image, as shown in FIG. 11. The second C-mode ultrasound image is generated based on the blood flow direction information and the blood flow velocity magnitude information of the blood vessel in the ROI, and is configured to show the blood flow direction information and the blood flow velocity magnitude information; and correspondingly, in Step 4, the processor 20 can generate a second multimodal ultrasound image based on the B-mode ultrasound image and the second C-mode ultrasound image, as shown in FIG. 12. Different colors are used to represent different blood flow directions. For example, if the projection of the true blood flow direction along the ultrasound beam direction is toward the probe, it is displayed in red (labeled as “Red” in FIG. 12); and if the projection is away from the probe, it is displayed in blue (labeled as “Blue” in FIG. 12). The correspondence between flow direction and color (red/blue) can be inverted as needed. The saturation or brightness of the colors represents the velocity magnitude, with darker red/blue indicating higher velocities in the figure. The third C-mode ultrasound image is generated based on the blood flow direction information and the blood flow energy information of the blood vessel in the ROI, and is configured to show the blood flow direction information and the blood flow energy information; and correspondingly, in Step 4, the processor 20 can generate a third multimodal ultrasound image based on the B-mode ultrasound image and the third C-mode ultrasound image, as shown in FIG. 13. Similarly, distinct blood flow directions are represented using different colors (consistent with FIG. 12), while color saturation or brightness indicates energy magnitude—in the figure, the saturation/brightness of red color (labeled as “Red” in FIG. 13) and blue color (labeled as “Blue” in FIG. 13) corresponds to energy levels.

The workflow illustrated in FIG. 5 represents the process from velocity scale acquisition to multimodal ultrasound image display. During clinical Doppler ultrasound examinations of patients, additional steps or operations may occur before and/or after the FIG. 5 workflow. Below is an example within the operational context of an ultrasound imaging device, as shown in FIG. 14. The ultrasound imaging device supports both the Doppler microvascular imaging mode and the Doppler conventional blood flow imaging mode, and its Doppler ultrasound imaging method comprises the following steps:

Step 0: The processor 20 displays the Doppler microvascular flow imaging mode and the Doppler conventional blood flow imaging mode on the display of the human-machine interface device for user selection. The human-machine interface device may also provide a physical button (e.g., dedicated or toggle buttons) to select one of these two imaging modes. Other selection mechanisms enabling user choice between the modes are also permissible. The Doppler microvascular imaging mode is preconfigured with a first velocity scale, and the Doppler conventional flow imaging mode is preconfigured with a second velocity scale. The first velocity scale is smaller than the second velocity scale.

Step 1: The processor 20 acquires the current velocity scale. Specifically, when the Doppler microvascular flow imaging mode is selected, the processor 20 retrieves the preconfigured first velocity scale of this mode; that is, after selecting the Doppler microvascular imaging mode, the preconfigured first velocity scale of the Doppler microvascular imaging mode is set as the current velocity scale.

Subsequent Steps 2-4 proceed as described previously and are not reiterated here.

Step 5: the processor 20 adjusts the current velocity scale. Following Step 4 in FIGS. 5 and 13, the current velocity scale may be further adjusted either manually or automatically. Exemplary implementations for both adjustment methods are provided below.

While the processor 20 displays the multimodal ultrasound image in real time on the display device, it simultaneously shows the current velocity scale. The velocity scale is adjustable and can be adjusted above 5 cm/s, meaning it has an adjustable range allowing users to select desired values within this range, with the maximum value of the range exceeding 5 cm/s. In some embodiments, considering that microvascular flow scenarios typically do not exceed 10 cm/s, the maximum adjustable range of the velocity scale may exceed 10 cm/s (i.e., adjustable above 10 cm/s). In other embodiments, for high-motion scenarios such as cardiac imaging, the maximum adjustable range can exceed 20 cm/s (i.e., adjustable above 20 cm/s). Users can adjust the velocity scale via an input unit on the human-machine interface, such as: rotating a dedicated velocity scale knob, or entering a new value directly on the display interface. The processor 20 receives, via the input unit, an instruction to adjust the current velocity scale, and updates the current velocity scale accordingly, thereby completing manual adjustment.

Automatic adjustment of the velocity scale can be achieved through multiple methods. For example: the processor 20 identifies the target tissue based on the B-mode ultrasound image and adjusts the current velocity scale to the predetermined velocity scale associated with that tissue. The first velocity scale has universal applicability for microvascular blood flow, while the predetermined velocity scale associated with the target tissue providestargeted specificity, allowing a two-layer selection mechanism to achieve an optimally matched final velocity scale. For another example, the processor 20 detects whether aliasing artifacts occur in the C-mode ultrasound image (e.g., via pattern recognition algorithms). If aliasing is present, it increases the current velocity scale by a preset increment to automatically resolve aliasing. For yet another example, the processor 20 acquires a plurality of predetermined velocity scales, controls the ultrasound probe to perform C-mode scanning on a ROI within the target tissue based on these predetermined velocity scales, respectively, and thereby obtains corresponding C-mode ultrasound image for each predetermined velocity scale (the specific process is detailed in Step 3 and not reiterated here). The processor 20 then evaluates the image quality (e.g., signal-to-noise ratio, artifact level) of corresponding C-mode ultrasound image for each predetermined velocity scale, and adjusts the current velocity scale to the predetermined velocity scale associated with the highest-quality C-mode ultrasound image, achieving optimal velocity scale matching.

Regardless of the adjustment method used for the velocity scale, the processor 20 controls the ultrasound probe to perform B-mode scanning on the target tissue and C-mode scanning on a ROI within the target tissue based on the adjusted velocity scale. It processes the echo of the ultrasound waves transmitted during the C-mode scanning to obtain the hemodynamic parameter of the blood vessel within the ROI, generates a C-mode ultrasound image showing the hemodynamic parameter based on the hemodynamic parameter of the blood vessel within the ROI, processes the echoes of ultrasound waves transmitted during the B-mode scanning to obtain a B-mode ultrasound image of the target tissue, and updates the real-time display of the multimodal ultrasound image based on the B-mode ultrasound image and the C-mode ultrasound image. In other words, after Step 5, the workflow returns to Step 1 to re-execute Steps 1-4, thereby continuously refreshing the multimodal ultrasound image on the display interface in real time.

In some embodiments, the Doppler microvascular imaging mode may further divided into an energy sub-mode and a velocity sub-mode. The preconfigured first velocity scale of the Doppler microvascular imaging mode may include a first sub-velocity scale associated with the energy sub-mode and a second sub-velocity scale associated with the velocity sub-mode. For example, in Step 0, the display interface may display the sub-modes (including the energy sub-mode and the velocity sub-mode) of the Doppler microvascular imaging mode for user selection. For another example, in Step 0, after selecting the Doppler microvascular imaging mode, the sub-modes become available for user selection. The processor 20 determines the sub-mode of the Doppler microvascular imaging mode based on the user's choice, where the first sub-velocity scale associated with the energy sub-mode is smaller than the second sub-velocity scale associated with the velocity sub-mode. Using a relatively smaller velocity scale for energy detection can enhance sensitivity of detection, while a higher velocity scale for velocity and direction measurement avoids aliasing artifacts. Correspondingly, in Step 1, the processor 20 determines the current sub-mode of the Doppler microvascular imaging mode. If the current sub-mode is the energy sub-mode, the processor 20 sets the first sub-velocity scale associated with the energy sub-mode as the current velocity scale. If the current sub-mode is the velocity sub-mode, the processor 20 sets the second sub-velocity scale associated with the velocity sub-mode as the current velocity scale.

In Step 3, when the sub-mode is determined as the energy sub-mode, the processor 20 processes the echoes of the non-focused ultrasound waves transmitted during the C-mode scanning to obtain the blood flow energy information of the blood vessel in the ROI, and further generates a first C-mode ultrasound image representing the blood flow energy information based on the blood flow energy information of the blood vessel in the ROI. Alternatively, when the sub-mode is determined as the energy sub-mode, the echoes of the non-focused ultrasound waves transmitted during the C-mode scanning are processed to obtain the blood flow direction information and the blood flow energy information of the blood vessel in the ROI; and a third C-mode ultrasound image representing the blood flow direction information and the blood flow energy information is generated based on the blood flow direction information and the blood flow energy information of the blood vessel in the ROI.

When the sub-mode is determined as the velocity sub-mode, the processor 20 processes the echoes of the non-focused ultrasound waves transmitted during the C-mode scanning to obtain the blood flow direction information and the blood flow velocity magnitude information of the blood vessel in the ROI, and generates a second C-mode ultrasound image representing the blood flow direction information and the blood flow velocity magnitude information based on the blood flow direction information and the blood flow velocity magnitude information of the blood vessel in the ROI.

In Step 4, when the sub-mode is determined as the energy sub-mode, the processor 20 generates a first multimodal ultrasound image based on the B-mode ultrasound image and the first C-mode ultrasound image and displays the first multimodal ultrasound image in real time; or, it generates a third multimodal ultrasound image based on the B-mode ultrasound image and the third C-mode ultrasound image, and displays the third multimodal ultrasound image in real time.

When the sub-mode is determined as the velocity sub-mode, the processor 20 generates a second multimodal ultrasound image based on the B-mode ultrasound image and the second C-mode ultrasound image, and displays the second multimodal ultrasound image in real time.

This embodiment demonstrates that under the energy sub-mode, where aliasing concerns are negligible, a lower velocity scale can be employed to achieve higher blood flow sensitivity; and under the velocity sub-mode, increasing the velocity scale appropriately avoids aliasing artifacts, thereby accurately reflecting the true velocity magnitude and direction of microvascular flow.

If the Doppler conventional blood flow imaging mode is selected by users-whether initially selecting it or switching from the Doppler microvascular imaging mode to the Doppler conventional flow imaging mode—the subsequent workflow remains structurally similar to the Doppler microvascular imaging mode but differs in Steps 2 and 3. Specifically, after selecting the Doppler conventional flow imaging mode, for example, the operational mode is switched from the Doppler microvascular imaging mode to the Doppler conventional flow imaging mode, the processor 20 acquires a preconfigured second velocity scale of the Doppler conventional flow imaging mode; that is, setting the preconfigured second velocity scale of the Doppler conventional flow imaging mode as the current velocity scale. Based on the current second velocity scale, the processor controls the ultrasound probe to perform B-mode scanning on the target tissue and C-mode scanning on a ROI within the target tissue. This controlling comprise: interleaving B-mode ultrasound image scanning during the scanning of every frame of C-mode ultrasound image. That is, under the Doppler conventional flow imaging mode, during the multiple samplings required to scan one frame of C-mode ultrasound image, B-mode ultrasound image scanning is interleaved during the intervals between at least two adjacent samplings. Specifically, B-mode ultrasound image scanning can be performed during the intervals between each ROI sampling for C-mode image acquisition. The ultrasound waves emitted during C-mode scanning may be focused waves, as described in FIG. 2 (not reiterated here). Further, the processor 20 processes the echoes of the focused waves emitted during C-mode scanning to obtain the hemodynamic parameter of the blood vessel within the ROI. The hemodynamic parameter comprises at least two of: blood flow direction information, blood flow energy information, and blood flow velocity magnitude information. Then the processor generates a C-mode ultrasound image showing the hemodynamic parameter based on the hemodynamic parameter of the blood vessel within the ROI, and processes the echoes of ultrasound waves transmitted during the B-mode scanning to obtain a B-mode ultrasound image of the target tissue. For example, the first implementation described in Step 3 can be specifically applied to process the echoes, thereby obtaining the hemodynamic parameter and the C-mode ultrasound image (details not reiterated here). The processor 20 generates and displays the multimodal ultrasound image in real time by combining the ultrasound B-mode image and C-mode image, thereby achieving conventional Doppler blood flow imaging.

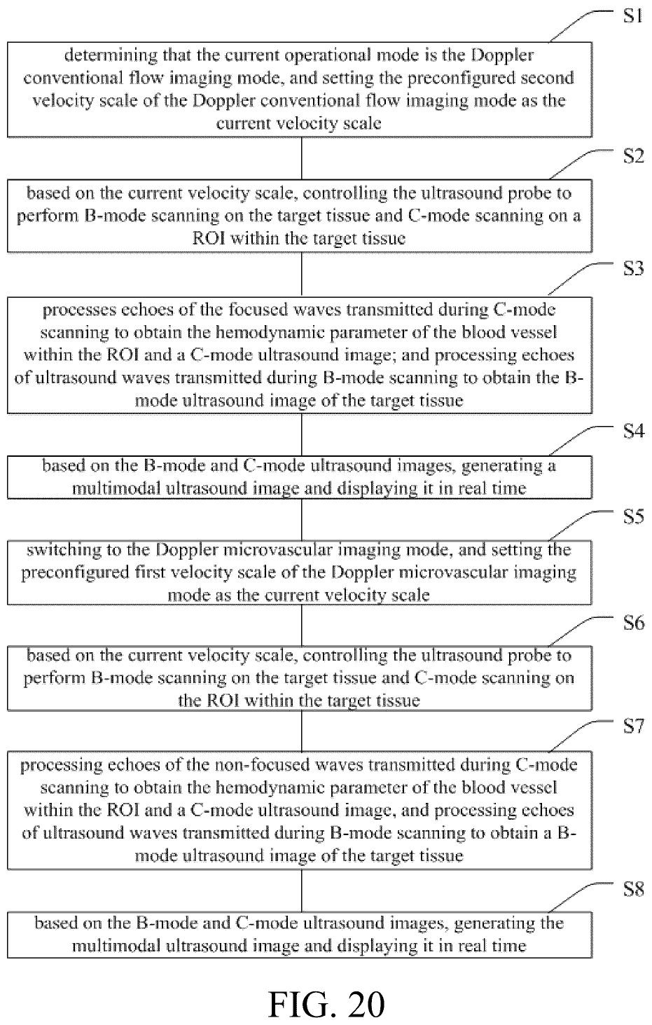

Some doctors prefer to initially assess larger blood vessels within the target tissue using the Doppler conventional blood flow imaging mode, then switch to microvascular evaluation if needed. The corresponding workflow, as illustrated in FIG. 20, comprises the following steps:

Step S1: the processor 20 determines that the current operational mode is the Doppler conventional flow imaging mode. For example, the processor 20 displays the Doppler microvascular imaging mode and the Doppler conventional flow imaging mode on the display of the human-machine interface device for user selection. The human-machine interface device may also provide a physical button (e.g., dedicated or toggle buttons) to select one of these two imaging modes. Other selection mechanisms enabling user choice between the modes are also permissible. The processor 20 receives, via the human-machine interface an instruction to select the Doppler conventional flow imaging mode, and setting the current operational mode as the Doppler conventional flow imaging mode in response to the instruction. Under the Doppler conventional flow imaging mode, the processor 20 sets the preconfigured second velocity scale of the Doppler conventional flow imaging mode as the current velocity scale.

Step S2: based on the current velocity scale (i.e., the second velocity scale, which may be adjusted from the second velocity scale by users), the processor 20 control the ultrasound probe to perform B-mode scanning on the target tissue and C-mode scanning on a ROI within the target tissue, wherein this control includes: interleaving B-mode ultrasound image scanning during the scanning of each frame of C-mode ultrasound image, and the ultrasound waves emitted during C-mode scanning are focused waves.

Step S3: the processor 20 processes the echoes of the focused waves emitted during C-mode scanning to obtain the hemodynamic parameter of the blood vessel within the ROI. The hemodynamic parameter includes at least two of: blood flow direction information, blood flow energy information, and blood flow velocity magnitude information. Based on the hemodynamic parameter of the blood vessel within the ROI, the processor generates a C-mode ultrasound image showing the hemodynamic parameter; and processes the echoes of ultrasound waves transmitted during the B-mode scanning to obtain the B-mode ultrasound image of the target tissue.

Step S4: based on the C-mode ultrasound image showing the hemodynamic parameter and the B-mode ultrasound image of the target tissue, the processor 20 generates a multimodal ultrasound image and displays it in real time. The specific implementations of Steps S1-S4 are detailed in the aforementioned embodiments and are not reiterated here.

Step S5: in response to an instruction to switch the operational mode to the Doppler microvascular imaging mode, the processor 20 switches the operational mode to the Doppler microvascular imaging mode. After the Doppler microvascular imaging mode is selected via the display device or using a physical button or other selection mechanisms by a user, the instruction to switch the operational mode to the Doppler microvascular imaging mode can be triggered. The processor 20 receives the instruction through the human-machine interface and executes it. Under the Doppler microvascular imaging mode, the processor 20 sets the preconfigured first velocity scale of the Doppler microvascular imaging mode as the current velocity scale.

Step S6: based on the current velocity scale, the processor 20 controls the ultrasound probe to perform B-mode scanning on the target tissue and C-mode scanning on a ROI within the target tissue. This control comprises: alternating scan sequences of at least one frame of B-mode ultrasound image followed by at least one frame of C-mode ultrasound image, or at least one frame of C-mode ultrasound image followed by at least one frame of B-mode ultrasound image; wherein the ultrasound waves emitted during the C-mode scanning are non-focused ultrasound waves.

Step S7: the processor 20 processes the echoes of the non-focused ultrasound waves transmitted during the C-mode scanning to obtain the hemodynamic parameter of the blood vessel within the ROI, the hemodynamic parameter comprising at least one of blood flow direction information, blood flow energy information, and blood flow velocity magnitude information; based on the hemodynamic parameter of the blood vessel within the ROI, the processor generates a C-mode ultrasound image showing the hemodynamic parameter, and processes the echoes of ultrasound waves transmitted during the B-mode scanning to obtain a B-mode ultrasound image of the target tissue.

Step S8: based on the C-mode ultrasound image showing the hemodynamic parameter and the B-mode ultrasound image of the target tissue, the processor 20 generates the multimodal ultrasound image and displays it in real time. Similarly, the specific implementations of Steps S5-S8 are detailed in the aforementioned embodiments and are not reiterated here. This Doppler ultrasound imaging method disclosed herein enables doctors to comprehensively assess both larger blood vessels and microvascular blood flow in patients.

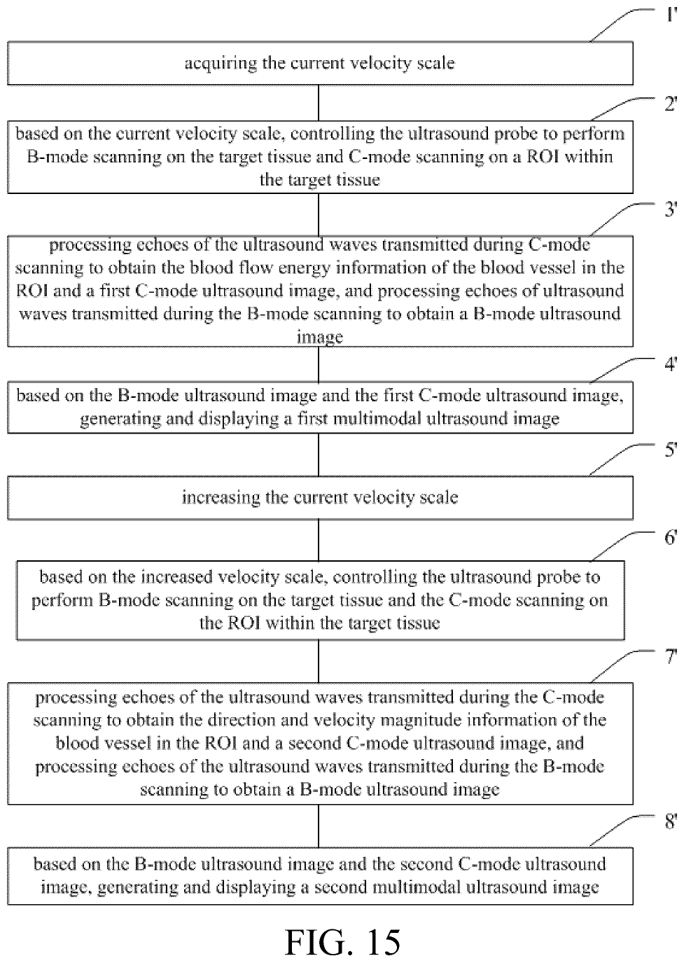

In some embodiments, when users need to detect blood flow energy without requiring directional information, the scanning mode involving B-mode and C-mode in the energy sub-mode may utilize existing interleaved B-mode with C-mode scanning instead of sequential B-mode and C-mode scanning (B-mode followed by C-mode or vice versa) to generate the first multimodal ultrasound image. Regardless of the method used to obtain the first multimodal ultrasound image, the processor 20 automatically increases the velocity scale upon user switching from the energy sub-mode to the velocity sub-mode, thereby meeting the requirements for the velocity sub-mode. The following example illustrates this embodiment. As shown in FIG. 15, in one application scenario, the Doppler ultrasound imaging process of the ultrasound imaging device comprises the following steps:

Step 1′: the processor 20 acquires the current velocity scale. For example, the ultrasound imaging device has the energy sub-mode and the velocity sub-mode for user selection. After selecting the energy sub-mode, the processor 20 acquires first sub-velocity scale associated with the energy sub-mode and sets it as the current velocity scale. The processor 20 may also receive user-input velocity scale and set it as the current velocity scale.

Step 2′: based on the current velocity scale, the processor 20 controls the ultrasound probe to perform B-mode scanning on the target tissue and C-mode scanning on a ROI within the target tissue. Specifically, as mentioned in above descriptions, interleaved scanning between B-mode and C-mode may be employed—that is, interleaving B-mode scanning during the intervals between each C-mode sampling of the ROI. Alternatively, sequential B-mode and C-mode scanning (as described in Step 2) can also be utilized, with details provided in the aforementioned Step 2 and not reiterated here. In summary, the scanning mode involving B-mode and C-mode is not limited to these specific implementations.

Step 3′: the processor 20 processes the echoes of the ultrasound waves emitted during C-mode scanning to obtain the blood flow energy information of the blood vessel in the ROI, generates a first C-mode ultrasound image representing the blood flow energy information based on the blood flow energy information of the blood vessel in the ROI, and processes the echoes of ultrasound waves transmitted during the B-mode scanning to obtain a B-mode ultrasound image of the target tissue. If interleaving B-mode scanning during C-mode scanning was employed in a previous step, this step utilizes a conventional processing method. If sequential B-mode and C-mode scanning was used in the previous step, the implementation aligns with Step 3 as previously described (not reiterated here).

Step 4′: based on the B-mode ultrasound image and the first C-mode ultrasound image, the processor 20 generates a first multimodal ultrasound image and displays it. For example, the processor 20 superimposes the B-mode ultrasound image onto the first C-mode ultrasound image to obtain a first multimodal ultrasound image and display it on the interface of the display device. In this way, users can visualize the blood flow energy information.

Step 5′: the processor 20 increases the current velocity scale. For example, a user may select the velocity sub-mode (i.e., switching from the energy sub-mode to the velocity sub-mode), and after the velocity sub-mode is selected, the processor 20 can acquire a second sub-velocity scale associated with the velocity sub-mode and set it as the current velocity scale, wherein the second sub-velocity scale associated with the velocity sub-mode is higher than the first sub-velocity scale associated with the energy sub-mode. The processor 20 may also increase the current velocity scale by a preset increment. These methods automatically elevate the velocity scale to meet the requirements for detecting blood flow velocity and direction in microvascular scenarios, which demand higher velocity scale settings.

Step 6′: based on the increased velocity scale, the processor 20 controls the ultrasound probe to perform B-mode scanning on the target tissue and the C-mode scanning on the ROI within the target tissue. This controlling comprises: alternating scans of at least one frame of B-mode ultrasound image followed by at least one frame of C-mode ultrasound image, or at least one frame of C-mode ultrasound image followed by at least one frame of B-mode ultrasound image; wherein the ultrasound waves emitted during C-mode scanning are non-focused ultrasound waves. The specific implementation aligns with Step 2 described above and is not reiterated here.

Step 7′: the processor 20 processes the echoes of the non-focused ultrasound waves transmitted during the C-mode scanning to obtain the blood flow direction information and the blood flow velocity magnitude information of the blood vessel in the ROI, generates a second C-mode ultrasound image representing the blood flow direction information and the blood flow velocity magnitude information based on the blood flow direction information and the blood flow velocity magnitude information of the blood vessel in the ROI, and processes the echoes of ultrasound waves transmitted during the B-mode scanning to obtain a B-mode ultrasound image. The specific implementation aligns with Step 3 described above and is not reiterated here.

Step 8′: based on the B-mode ultrasound image and the second C-mode ultrasound image, the processor 20 generates a second multimodal ultrasound image and displays it in real time. For example, the processor 20 superimposes the B-mode ultrasound image onto the second C-mode ultrasound image to obtain a second multimodal ultrasound image and displays it in real time on the interface of the display device, thereby enabling users to visualize the velocity and direction data of blood flow.

The Doppler microvascular flow imaging mode in conventional implementations of the prior art can only detect blood flow energy. Since vascular pathways of microvessels can be visualized through energy information, practitioners in this technical field has shown minimal interest in enhancing microvascular scenarios to achieve detection of velocity and direction. In contrast, the present disclosure-whether employing the aforementioned methods or alternative approaches-enables detection of at least two parameters among blood flow direction information, blood flow energy information, and blood flow velocity magnitude information, representing significant advancement over prior art limited to energy detection alone. The following describes an exemplary embodiment.

In some embodiments, the processor 20 controls the ultrasound probe to perform B-mode scanning on the target tissue and C-mode scanning on a ROI within the target tissue. For example, it may acquire the current velocity scale, and then based on the current velocity scale, it controls the ultrasound probe to perform B-mode scanning on the target tissue and C-mode scanning on a ROI within the target tissue.

Further, the processor 20 processes the echoes of the ultrasound waves emitted during C-mode scanning to obtain the hemodynamic parameter of the blood vessel within the ROI. The hemodynamic parameter comprises at least two of: blood flow direction information, blood flow energy information, and blood flow velocity magnitude information. Regardless of the scanning mode involving B-mode and C-mode (sequential or interleaved) or echo processing techniques employed, the device ensures acquisition of at least two of these parameters. The specific methods for deriving these hemodynamic parameters are detailed in the aforementioned embodiments and are not restated here.

The processor 20 generates a C-mode ultrasound image showing the hemodynamic parameter based on the hemodynamic parameter of the blood vessel within the ROI. The C-mode ultrasound image includes at least one of a first C-mode ultrasound image, a second C-mode ultrasound image, and a third C-mode ultrasound image. For example, the C-mode ultrasound image includes the first C-mode ultrasound image and the second C-mode ultrasound image. For another example, the C-mode ultrasound image includes the second C-mode ultrasound image and/or third C-mode ultrasound image. Similarly, the processor 20 processes the echoes of ultrasound waves transmitted during the B-mode scanning to obtain a B-mode ultrasound image. The generation of the first, second, and third C-mode ultrasound images are exemplified in the preceding embodiments and are not reiterated here.

Based on the B-mode ultrasound image and the C-mode ultrasound image, the processor 20 generates a multimodal ultrasound image and displays it. For example, the two types of images are overlaid to produce the multimodal ultrasound image, which is then displayed on the display device. The generated multimodal ultrasound image includes at least one of a first multimodal ultrasound image, a second multimodal ultrasound image and a third multimodal ultrasound image. That is the type of multimodal ultrasound image generated depends on or corresponds to the previously obtained C-mode ultrasound image. For instance, the generated multimodal ultrasound image may comprises the first multimodal ultrasound image and the second multimodal ultrasound image; or it may include the second multimodal ultrasound image and/or the third multimodal ultrasound image. The generation of the first, second, and third multimodal ultrasound images are exemplified in the preceding embodiments and are not reiterated here.

Following the generation of multimodal ultrasound images through the aforementioned embodiments, spectral analysis may be further performed. The multimodal ultrasound images obtained via these methods enable visualization of microvascular flow, including its velocity and direction, thereby allowing users to conduct spectral analysis on such flow. The following embodiments provide illustrative examples.

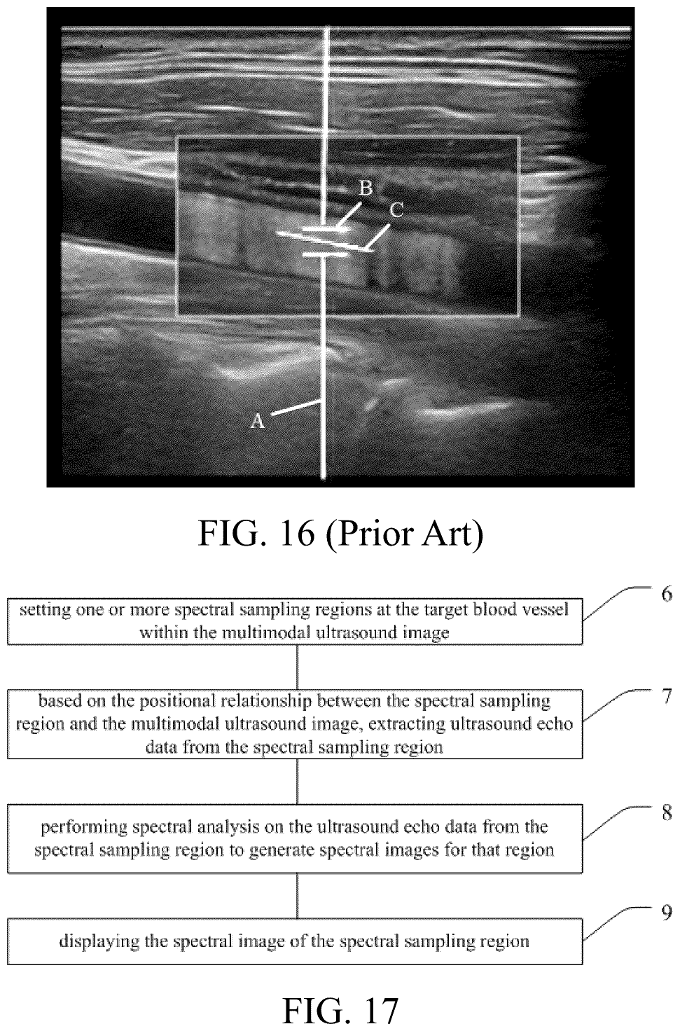

Under spectral Doppler mode, a sample volume (SV) is manually set by users on the target vessel within the ultrasound image through human-machine interaction to obtain spectral Doppler measurements at the SV site. Under current spectral Doppler mode, as shown in FIG. 16, when setting the sample volume, a long sampling line A is displayed. Two short horizontal lines B are positioned on the sampling line A, with an oblique line C placed between the two short lines B. The sampling line A indicates the beam direction of the ultrasound scan. The region between the two short horizontal lines B defines the sample volume, which also includes the oblique line C to represent the actual blood flow direction of the target vascular structure, thereby providing a correction angle required for spectral analysis. However, this conventional sample volume configuration is constrained by the ultrasound beam orientation range: (i) The angular adjustment of sampling line A is limited, restricting the positional adaptability of the sample volume. (ii) The two short lines B form a fixed-shape sample volume, with their separation adjustable only in predefined steps within a narrow range. Given the diversity of vascular anatomies (including variations in position, size, shape, and flow direction), this rigid sample volume format cannot achieve precise alignment with target vessels, directly compromising the accuracy of subsequent spectral quantitative analyses.

In contrast, the sample volume in the present disclosure is not constrained by these limitations and can be adaptively aligned with vascular contours, thereby improving the accuracy of spectral quantitative analysis. Multiple specific implementations exist, two of which are exemplarily described below.

In one implementation, as shown in FIG. 17, after displaying the multimodal ultrasound image, the processor 20 may execute Steps 6-8. Specifically, the processor 20 can receive a user-initiated instruction to enter spectral Doppler mode and, in response to this instruction, activate the spectral Doppler mode.

Under spectral Doppler mode, the processor 20 controls the ultrasound probe to transmit ultrasound waves toward the ROI within the target tissue. Ultrasound echo data is derived from the reflected echoes, where the transmitted waves may be focused waves or non-focused waves, such as plane waves or divergent waves. In some embodiments, the ultrasound echo data may alternatively correspond to the ultrasound C-mode image data, i.e., the ultrasound echoes obtained in the aforementioned Steps 2 and 3.

Step 6: The processor 20 sets one or more spectral sampling regions at the target blood vessel within the multimodal ultrasound image. These spectral sampling regions are defined as regions from which ultrasound echo data is subsequently acquired to generate spectral images at those regions.

In some embodiments, the ultrasound echo data within the set spectral sampling regions includes ultrasound echo data from multiple receiving scan lines. Compared to existing methods where spectral sampling regions are arranged along a single scan line (wherein the conventional spectral sampling regions can only include one scan line, thus yielding echo data limited to that single scan line within the region), the acquired data volume is insufficient to cover the width of larger spectral sampling regions. In contrast, spectral sampling regions in the present embodiment incorporate ultrasound echo data corresponding to multiple receiving scan lines, thereby obtaining more comprehensive echo data within the spectral sampling region, which enhances the accuracy of subsequent spectral analyses.

In other embodiments, at least one of the spectral sampling regions has an angle between its major axis and the beam propagation direction of ultrasound waves transmitted into that region. The major axis of a spectral sampling region is defined as the axis connecting the two farthest boundary points within the region. For example: If the spectral sampling region is rectangular, the major axis aligns with the rectangle's length. If elliptical, the major axis follows the anatomical major axis. If irregularly shaped, multiple major axes may exist (e.g., in polygonal regions). The beam propagation direction refers to the transmission direction of ultrasound waves sent into the spectral sampling region, or the extension direction of receiving scan lines corresponding to the ultrasound echo data in that region. Compared to existing methods where the major axis of a spectral sampling region must strictly align with the sampling line direction, the present embodiment allows diverse and flexible configurations of spectral sampling regions.

In some embodiments, the spectral sampling region comprises a closed region and a first straight line traversing the closed region, wherein: the size of the closed region matches the dimensions of the target vessel; the shape of the closed region corresponds to the shape of the target vessel; and the extension direction of the first straight line aligns with the blood flow direction within the target vessel. In the present embodiment, the shape of the closed region is not fixed and may include rectangles, ellipses, or irregular geometries, which can be identical to or infinitely approximate the shape of the target vessel. The size of the closed region refers to the spatial dimensions of the spectral sampling region, which may match or closely approximate the cross-sectional region of the target vessel. In microvascular scenarios, conventional spectral sampling regions (e.g., region D1 in FIG. 19) fail to precisely align with the target vessel requiring spectral analysis. As shown in FIG. 19, conventional spectral sampling region D1 erroneously includes two vessels with opposing flow directions. Spectral analysis of echo data from region D1 generates a spectrogram displaying both positive and negative flow velocities, preventing users from isolating the hemodynamic profile of individual vessels. In contrast, the spectral sampling regions proposed in the present disclosure (e.g., regions SV1, SV2, SV3, and SV4 in FIG. 19) achieve sub-vessel precision. For example, spectral sampling region SV2 exclusively encapsulates a single microvessel.

In the present embodiment, the spectral sampling region may be: manually configured by the user on the multimodal ultrasound image, or automatically configured by identifying information such as the position of the target vessel within the multimodal ultrasound image, or configured through a hybrid approach combining these automatic and manual methods. The following illustrates on each setting of the spectral sampling regions separately.

In some embodiments, the spectral sampling region may be manually set by a user on the multimodal ultrasound image. By observing the blood flow morphology in the multimodal ultrasound image, the user selects the target vessel for spectral measurement based on the blood flow morphology. For example, an instruction to set the spectral sampling region may be inputted by the user via the input unit such as a mouse, keyboard, or trackball. In response to the instruction to set the spectral sampling region, the spectral sampling region is displayed at a default initial position on the multimodal ultrasound image. Then, through the input unit, the user inputs adjustment operations for the spectral sampling region, that is, manually adjusts one or more of the following in the spectral sampling region: the position of the closed region, the shape of the closed region, the size of the closed region, and the extension direction of the first straight line, so that the contour of the adjusted spectral sampling region matches the contour of the target vessel displayed in the multimodal ultrasound image. Manual adjustment may include one or more of the following: adjusting the position of the spectral sampling region to place the spectral sampling region at the target vessel; adjusting the shape and/or size of the closed region in the spectral sampling region to match the shape and/or size of the target vessel; rotating the extension direction of the first straight line in the spectral sampling region to align with the blood flow direction in the target vessel. Additionally, during manual setup, the multimodal ultrasound image may display not only medium and large vessels but also microvessels with smaller diameters. Therefore, to enable more precise setting of the spectral sampling region, the user is permitted to magnify a local area of the multimodal ultrasound image, which aids in observing fine blood flow details. On the magnified multimodal ultrasound image, the spectral sampling region SV2 for spectral analysis is selected. The closed region of this spectral sampling region is rectangular, and the first straight line indicates the current blood flow direction. The height and width of the spectral sampling region are adjusted to match the contour of the target vessel, and the spectral sampling region is rotated to align the first straight line with the blood flow direction of the target vessel.

In other embodiments, the spectral sampling region may be automatically configured, which includes: in response to an instruction (inputted via the input unit such as a mouse, keyboard, or trackball by the user) to set spectral sampling region, performing blood flow analysis on the multimodal ultrasound image to identify the region with the strongest blood flow signal as the target vessel; obtaining the position information of the target vessel to further determine the size, shape, and flow direction of the target vessel based on the blood flow signal; and finally, based on one or more of the position information, size, shape, and flow direction of the target vessel, automatically generating the spectral sampling region that matches the contour of the target vessel displayed in the multimodal ultrasound image, and displaying the spectral sampling region on the image.

In yet other embodiments, the spectral sampling region can be configured through a combined manual and automatic approach, which includes: in response to an instruction (inputted via the input unit such as a mouse, keyboard, or trackball by the user) to set spectral sampling region, initiating the configuration workflow of the spectral sampling region; selecting a region on the multimodal ultrasound image via the input unit (e.g., the mouse, keyboard, trackball, etc.) by the user as the target blood vessel to further acquire the position information of the selected target vessel; performing blood flow analysis on the target blood vessel in the multimodal ultrasound image to determine its size, shape, and flow direction; and finally, based on one or more of the position information, size, shape, and flow direction of the target vessel, automatically generating a spectral sampling region that matches the contour of the target vessel displayed in the multimodal ultrasound image and displaying the spectral sampling region on the multimodal ultrasound image.

In some embodiments, the spectral sampling region(s) may be singular or multiple. When multiple spectral sampling regions are configured on the multimodal ultrasound image, they may be configured on different segments of a single vessel for sampling, or configured on distinct vessels for independent sampling.

Step 7: The processor 20 extracts ultrasound echo data at the spectral sampling region based on the positional relationship between the spectral sampling region and the multimodal ultrasound image. After configuring the spectral sampling region within the multimodal ultrasound image, the positional information of the spectral sampling region within the multimodal ultrasound image is determined. Based on this positional information, the processor 20 extracts echo data specific to the spectral sampling region from the ultrasound echo data acquired in Step 6, which constitutes the data for spectral analysis.

Step 8: The processor 20 performs spectral analysis on the ultrasound echo data at the spectral sampling region to generate spectral images for that region. The processor performs spectral analysis on the extracted data to obtain spectral images such as blood flow spectrograms. The spectral analysis may include any one or more spectral analysis methods operable in spectral Doppler mode, which this embodiment does not specifically limit.

Step 9: The processor 20 displays the spectral image of the spectral sampling region on the display interface of the display device. In some embodiments, when multiple spectral sampling regions are configured in the multimodal ultrasound image: the corresponding spectral images of the multiple spectral sampling regions can be displayed simultaneously; or only the spectral image corresponding to a selected spectral sampling region can be displayed.

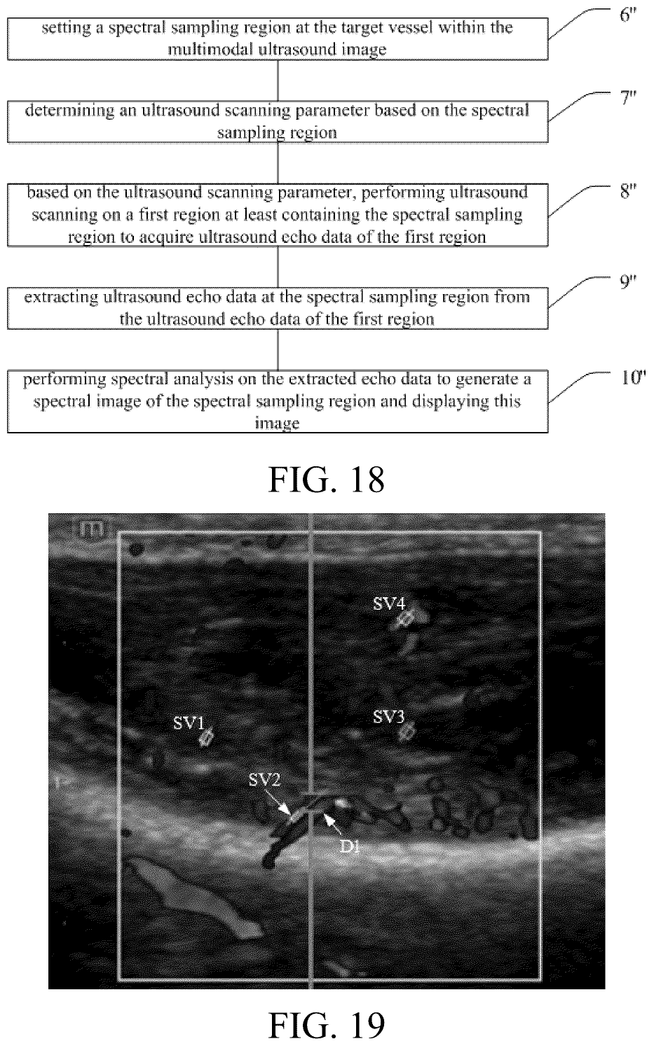

Alternative Implementation, as shown in FIG. 18, comprises the following steps:

Step 6″: The processor 20 sets a spectral sampling region at the target vessel within the multimodal ultrasound image. Here, the spectral sampling region is defined as an region for subsequent sampling of ultrasound echo data within that region to calculate Doppler spectral results. Specific implementations align with the aforementioned Step 6 and are not reiterated here.

Step 7″: The processor 20 determines ultrasound scanning parameters based on the spectral sampling region. In this embodiment, after establishing the spectral sampling region in Step 6″, parameters such as the region's position, size, and orientation are used to adjust the ultrasound scanning parameters of the imaging device. This adjustment ensures the device transmits ultrasound waves toward the smallest region (first region) encompassing the spectral sampling region.

Step 8″: based on the ultrasound scanning parameter, the processor 20 performs ultrasound scanning on a first region at least containing the spectral sampling region to acquire ultrasound echo data of the first region. In some embodiments, the first region may exactly coincide with the spectral sampling region. Alternatively, it may be the smallest region covering and adjacent to the spectral sampling region.

Step 9″: The processor 20 extracts ultrasound echo data specific to the spectral sampling region from the ultrasound echo data of the first region.

Step 10″: The processor 20 performs spectral analysis on the extracted echo data to generate a spectral image of the spectral sampling region and displays this image on the display.

Accordingly, under spectral Doppler mode, configuring the spectral sampling region at the target vessel within the multimodal ultrasound image ensures that the ultrasound echo data within the spectral sampling region includes ultrasound echo data from multiple receive scan lines. Performing spectral analysis on this echo data enables more precise acquisition of spectral Doppler measurements for the target vessel.

The present disclosure refers to various exemplary embodiments for illustrative purposes. However, those skilled in the art will recognize that modifications and alterations may be made to these embodiments without departing from the scope of the disclosure. For instance, individual operational steps and components for performing such steps may be implemented in diverse manners depending on specific applications or considerations of cost functions associated with system operations (e.g., one or more steps may be deleted, modified, or consolidated with other steps).

Moreover, as understood by those skilled in the art, the principles disclosed may be embodied in a computer program product stored on a non-transitory computer-readable storage medium preloaded with computer-readable program code. Any tangible, non-transitory computer-readable storage medium may be utilized, including but not limited to: magnetic storage devices (e.g., hard disks, floppy disks); optical storage devices (e.g., CD-ROMs, DVDs, Blu-ray discs); and flash memory devices. The computer program instructions may be loaded onto a general-purpose computer, special-purpose computer, or other programmable data processing apparatus to create a machine, such that the instructions executed on the computer or programmable apparatus produce means for implementing specified functions. These instructions may also reside in a computer-readable memory, directing the computer or programmable apparatus to operate in a defined manner, thereby forming an article of manufacture comprising functional implementation means. Furthermore, the computer program instructions may be executed on a computer or programmable data processing apparatus to generate a computer-implemented process, wherein the executed instructions provide steps for realizing the specified functionality, including but not limited to: technical improvements in data processing efficiency (e.g., optimized memory allocation); and enhanced accuracy in algorithmic execution (e.g., reduced error margins in machine learning models).

While the principles disclosed herein have been illustrated through various embodiments, it should be understood that structural configurations, material selections, and component proportions particularly suited to specific operational environments may be modified without departing from the scope and spirit of the disclosure. Such modifications, along with other adaptations or adjustments, shall be encompassed within the scope of the present disclosure.

The foregoing detailed description has been described with reference to various embodiments. However, those skilled in the art will recognize that modifications and variations may be made without departing from the scope of the disclosure. Accordingly, the description of the disclosure shall be interpreted in an illustrative rather than restrictive sense, and all such modifications are intended to be included within its scope. Similarly, discussions of advantages, alternative solutions to problems, and operational benefits associated with the embodiments are provided above. Nevertheless, benefits, advantages, solutions to problems, and any elements that may produce such effects or render them more explicit shall not be construed as critical, required, or essential. Furthermore, the term ‘coupled’ and its derivatives encompass physical connections (e.g., mechanical joints), electrical connections (e.g., circuit interconnects), magnetic linkages (e.g., inductive coupling), optical interfaces (e.g., fiber-optic alignment), communication channels (e.g., wireless protocols), functional integrations (e.g., software APIs), and any other form of association that achieves operational interaction.

Those skilled in the art will recognize that numerous modifications to the details of the above-described embodiments may be made without departing from the fundamental principles of the disclosed subject matter. Accordingly, the scope of the present disclosure shall be determined solely by the claimsand their legal equivalents.

What is claimed is:

1. A Doppler ultrasound imaging method for an ultrasound imaging device, comprising:
acquiring a current velocity scale;
controlling an ultrasound probe, based on the current velocity scale, to perform B-mode scanning on a target tissue and C-mode scanning on a region of interest within the target tissue, comprising alternately performing scanning by:
at least one frame of B-mode ultrasound image followed by at least one frame of C-mode ultrasound image, or
at least one frame of C-mode ultrasound image followed by at least one frame of B-mode ultrasound image; wherein non-focused ultrasound waves are transmitted during the C-mode scanning;
processing echoes of the non-focused ultrasound waves transmitted during the C-mode scanning to obtain a hemodynamic parameter of a blood vessel in the region of interest, said hemodynamic parameter comprising at least one of:
blood flow direction information,
blood flow energy information, and
blood flow velocity magnitude information; and
generating a C-mode ultrasound image showing the hemodynamic parameter, based on the hemodynamic parameter of the blood vessel within the region of interest;
processing echoes of ultrasound waves transmitted during the B-mode scanning to obtain a B-mode ultrasound image of the target tissue;
generating a multimodal ultrasound image based on the C-mode ultrasound image showing the hemodynamic parameter and the B-mode ultrasound image of the target tissue, and
displaying the multimodal ultrasound image in real time,
wherein at least one blood vessel in the C-mode ultrasound image showing the hemodynamic parameter has a diameter less than or equal to 200 micrometers.

2. The method according to claim 1, wherein:
the C-mode ultrasound image showing the hemodynamic parameter comprises at least one of:
a first C-mode ultrasound image, generated based on the blood flow energy information of the blood vessel in the region of interest and configured to show the blood flow energy information;
a second C-mode ultrasound image, generated based on the blood flow direction information and the blood flow velocity magnitude information of the blood vessel in the region of interest and configured to show the blood flow direction information and the blood flow velocity magnitude information; and
a third C-mode ultrasound image, generated based on the blood flow direction information and the blood flow energy information of the blood vessel in the region of interest and configured to show the blood flow direction information and the blood flow energy information;
and,
the multimodal ultrasound image comprises at least one of:
a first multimodal ultrasound image, generated based on the B-mode ultrasound image of the target tissue and the first C-mode ultrasound image;
a second multimodal ultrasound image, generated based on the B-mode ultrasound image of the target tissue and the second C-mode ultrasound image; and
a third multimodal ultrasound image generated based on the B-mode ultrasound image of the target tissue and the third C-mode ultrasound image.

3. The method according to claim 1, wherein
when displaying the multimodal ultrasound image in real time, the current velocity scale is concurrently displayed and is adjustable to be greater than 5 cm/s.

4. The method according to claim 1, wherein
acquiring the current velocity scale comprises:
obtaining a predetermined velocity scale associated with the target tissue, and setting the predetermined velocity scale associated with the target tissue as the current velocity scale; or
setting a user-input velocity scale as the current velocity scale.

5. The method according to claim 3, further comprising:
identifying the target tissue based on the B-mode ultrasound image of the target tissue, and adjusting the current velocity scale to the predetermined velocity scale associated with the target tissue; or,
in response to an instruction to adjust the current velocity scale, adjusting the current velocity scale.

6. The method according to claim 3, further comprising:
determining whether aliasing artifacts are present in the C-mode ultrasound image showing the hemodynamic parameter, and if present, increasing the current velocity scale;
or,
acquiring a plurality of different predetermined velocity scales,
controlling the ultrasound probe to perform C-mode scanning on the region of interest within the target tissue according to each of the predetermined velocity scales, respectively, thereby obtaining a corresponding C-mode ultrasound image for each of the predetermined velocity scales;
assessing an image quality of the corresponding C-mode ultrasound image for each of the predetermined velocity scales, and
adjusting the current velocity scale to a predetermined velocity scale corresponding to a C-mode ultrasound image with a highest image quality.

7. The method according to claim 5, further comprising:
controlling the ultrasound probe, based on the adjusted velocity scale, to perform B-mode scanning on the target tissue and C-mode scanning on the region of interest within the target tissue, comprising alternately performing scanning by:
at least one frame of B-mode ultrasound image followed by at least one frame of C-mode ultrasound image, or
at least one frame of C-mode ultrasound image followed by at least one frame of B-mode ultrasound image;
wherein non-focused ultrasound waves are transmitted during said C-mode scanning;
processing echoes of said non-focused ultrasound waves transmitted during said C-mode scanning to obtain a hemodynamic parameter of the blood vessel within the region of interest, and generating a C-mode ultrasound image showing said hemodynamic parameter based on said hemodynamic parameter of the blood vessel within the region of interest;
processing echoes of ultrasound waves transmitted during said B-mode scanning to obtain a B-mode ultrasound image of the target tissue; and
updating real-time display of the multimodal ultrasound image based on said C-mode ultrasound image showing the hemodynamic parameter and said B-mode ultrasound image of the target tissue.

8. The method according to claim 6, further comprising:
controlling the ultrasound probe, based on the adjusted velocity scale, to perform B-mode scanning on the target tissue and C-mode scanning on the region of interest within the target tissue, comprising alternately performing scanning by:
at least one frame of B-mode ultrasound image followed by at least one frame of C-mode ultrasound image, or
at least one frame of C-mode ultrasound image followed by at least one frame of B-mode ultrasound image;
wherein non-focused ultrasound waves are transmitted during said C-mode scanning;
processing echoes of said non-focused ultrasound waves transmitted during said C-mode scanning to obtain a hemodynamic parameter of the blood vessel within the region of interest, and generating a C-mode ultrasound image showing said hemodynamic parameter based on said hemodynamic parameter of the blood vessel within the region of interest;
processing echoes of ultrasound waves transmitted during said B-mode scanning to obtain a B-mode ultrasound image of the target tissue; and
updating real-time display of the multimodal ultrasound image based on said C-mode ultrasound image showing the hemodynamic parameter and said B-mode ultrasound image of the target tissue.

9. The method according to claim 1, wherein:
before acquiring the current velocity scale, the method further comprises:
displaying a Doppler microvascular imaging mode and a Doppler conventional flow imaging mode for user selection, wherein the Doppler microvascular imaging mode is preconfigured with a first velocity scale, the Doppler conventional flow imaging mode is preconfigured with a second velocity scale, and the first velocity scale is lower than the second velocity scale;
said acquiring the current velocity scale comprises:
setting the preconfigured first velocity scale of the Doppler microvascular imaging mode as the current velocity scale after the Doppler microvascular imaging mode is selected.

10. The method according to claim 9, wherein
the Doppler microvascular imaging mode comprises an energy sub-mode and a velocity sub-mode,
the preconfigured first velocity scale of the Doppler microvascular imaging mode comprises:
a first sub-velocity scale associated with the energy sub-mode, and
a second sub-velocity scale associated with the velocity sub-mode,
wherein the first sub-velocity scale associated with the energy sub-mode is lower than the second sub-velocity scale associated with the velocity sub-mode;
said setting the preconfigured first velocity scale of the Doppler microvascular imaging mode as the current velocity scale after the Doppler microvascular imaging mode is selected, comprises:
determining a current sub-mode of the Doppler microvascular imaging mode;
when the current sub-mode is the energy sub-mode, setting the first sub-velocity scale associated with the energy sub-mode as the current velocity scale; and
when the current sub-mode is the velocity sub-mode, setting the second sub-velocity scale associated with the velocity sub-mode as the current velocity scale;
said processing echoes of the non-focused ultrasound waves transmitted during the C-mode scanning to obtain a hemodynamic parameter of the blood vessel within the region of interest, and generating a C-mode ultrasound image showing the hemodynamic parameter based on the hemodynamic parameter of the blood vessel within the region of interest, comprises:
(i) when the current sub-mode is the energy sub-mode,
processing the echoes of the non-focused ultrasound waves transmitted during the C-mode scanning to obtain blood flow energy information of the blood vessel in the region of interest, or to obtain blood flow direction information and blood flow energy information of the blood vessel in the region of interest; and
generating a first C-mode ultrasound image showing the blood flow energy information based on the blood flow energy information of the blood vessel in the region of interest, or generating a third C-mode ultrasound image showing the blood flow direction information and the blood flow energy information based on the blood flow direction information and the blood flow energy information of the blood vessel in the region of interest; and
(ii) when the current sub-mode is the velocity sub-mode,
processing the echoes of the non-focused ultrasound waves transmitted during the C-mode scanning to obtain blood flow direction information and blood flow velocity magnitude information of the blood vessel in the region of interest, and
generating a second C-mode ultrasound image showing the blood flow direction information and the blood flow velocity magnitude information based on the blood flow direction information and the blood flow velocity magnitude information of the blood vessel in the region of interest;
said generating a multimodal ultrasound image based on the B-mode ultrasound image of the target tissue and the C-mode ultrasound image showing the hemodynamic parameter, and displaying the multimodal ultrasound image in real time, comprises:
(i) when the current sub-mode is the energy sub-mode,
generating a first multimodal ultrasound image based on the B-mode ultrasound image of the target tissue and the first C-mode ultrasound image, and displaying the first multimodal ultrasound image in real time; or
generating a third multimodal ultrasound image based on the B-mode ultrasound image of the target tissue and the third C-mode ultrasound image, and displaying the third multimodal ultrasound image in real time; and
(ii) when the current sub-mode is the velocity sub-mode,
generating a second multimodal ultrasound image based on the B-mode ultrasound image of the target tissue and the second C-mode ultrasound image, and displaying the second multimodal ultrasound image in real time.

11. The method according to claim 9, further comprising:
setting the preconfigured second velocity scale of the Doppler conventional flow imaging mode as the current velocity scale after the Doppler conventional flow imaging mode is selected;
controlling the ultrasound probe, based on the current velocity scale, to perform B-mode scanning on the target tissue and C-mode scanning on the region of interest within the target tissue, comprising:
interleaving B-mode ultrasound image scanning during scanning of each frame of C-mode ultrasound image,
wherein focused ultrasound waves are transmitted during said C-mode scanning;
processing echoes of the focused waves transmitted during said C-mode scanning to obtain a hemodynamic parameter of the blood vessel within the region of interest, said hemodynamic parameter comprising at least two of:
blood flow direction information,
blood flow energy information, and
blood flow velocity magnitude information;
generating a C-mode ultrasound image showing said hemodynamic parameter, based on said hemodynamic parameter of the blood vessel within the region of interest,
processing echoes of ultrasound waves transmitted during said B-mode scanning to obtain a B-mode ultrasound image of the target tissue;
generating a multimodal ultrasound image based on said C-mode ultrasound image showing said hemodynamic parameter and said B-mode ultrasound image of the target tissue, and displaying said multimodal ultrasound image in real time.

12. The method according to claim 11, wherein
said controlling the ultrasound probe, based on the current velocity scale, to perform B-mode scanning on the target tissue and C-mode scanning on the region of interest within the target tissue, further comprises:
performing a plurality of samplings on the region of interest to scan a frame of C-mode ultrasound image, wherein a sampling period corresponding to each sampling is inversely proportional to the current velocity scale; wherein:
under the Doppler conventional flow imaging mode, while performing the plurality of samplings on the region of interest to scan the frame of C-mode ultrasound image, said B-mode ultrasound image scanning is intermittently interleaved between at least two adjacent samplings; and
under the Doppler microvascular imaging mode, the plurality of samplings on the region of interest to scan the frame of C-mode ultrasound image are performed in absence of said B-mode ultrasound image scanning.

13. A Doppler ultrasound imaging method for an ultrasound imaging device, comprising:
acquiring a current velocity scale;
controlling an ultrasound probe, based on the current velocity scale, to perform B-mode scanning on a target tissue and C-mode scanning on a region of interest within the target tissue;
processing echoes of ultrasound waves transmitted during the C-mode scanning to obtain blood flow energy information of a blood vessel in the region of interest, and generating a first C-mode ultrasound image showing the blood flow energy information based on the blood flow energy information of the blood vessel in the region of interest;
processing echoes of ultrasound waves transmitted during the B-mode scanning to obtain a B-mode ultrasound image of the target tissue;
generating a first multimodal ultrasound image based on the first C-mode ultrasound image and the B-mode ultrasound image, and displaying the first multimodal ultrasound image in real time;
increasing the current velocity scale;
controlling the ultrasound probe, based on the increased velocity scale, to perform B-mode scanning on the target tissue and C-mode scanning on the region of interest within the target tissue, comprising alternately performing scanning by:
at least one frame of B-mode ultrasound image followed by at least one frame of C-mode ultrasound image, or
at least one frame of C-mode ultrasound image followed by at least one frame of B-mode ultrasound image;
wherein non-focused ultrasound waves are transmitted during said C-mode scanning;
processing echoes of the non-focused ultrasound waves transmitted during said C-mode scanning to obtain blood flow direction information and blood flow velocity magnitude information of the blood vessel in the region of interest, and
generating a second C-mode ultrasound image showing the blood flow direction information and the blood flow velocity magnitude information, based on the blood flow direction information and the blood flow velocity magnitude information of the blood vessel in the region of interest;
processing echoes of ultrasound waves transmitted during said B-mode scanning to obtain a B-mode ultrasound image of the target tissue; and
generating a second multimodal ultrasound image based on the second C-mode ultrasound image and said B-mode ultrasound image of the target tissue, and
displaying the second multimodal ultrasound image in real time.

14. The method according to claim 12, further comprising:
transmitting ultrasound waves to the region of interest within the target tissue, and acquiring ultrasound echo data based on echoes of said ultrasound waves; or, acquiring ultrasound echo data based on the echoes of the ultrasound waves transmitted during the C-mode scanning;
configuring at least one spectral sampling region at a target blood vessel in the multimodal ultrasound image, such that ultrasound echo data within the spectral sampling region contains ultrasound echo data from a plurality of receiving scan lines;
extracting the ultrasound echo data within the spectral sampling region from the ultrasound echo data based on a positional relationship between the spectral sampling region and the multimodal ultrasound image, wherein the extracted ultrasound echo data within the spectral sampling region contains the ultrasound echo data from the plurality of receiving scan lines;
performing spectral analysis on the extracted the ultrasound echo data within the spectral sampling region to obtain a spectral image at the spectral sampling region; and
displaying the spectral image.

15. The method according to claim 1, further comprising:
transmitting ultrasound waves to the region of interest within the target tissue, and acquiring ultrasound echo data based on echoes of said ultrasound waves; or, acquiring ultrasound echo data based on the echoes of the ultrasound waves transmitted during the C-mode scanning;
configuring at least one spectral sampling region at a target blood vessel in the multimodal ultrasound image, such that ultrasound echo data within the spectral sampling region contains ultrasound echo data from a plurality of receiving scan lines;
extracting the ultrasound echo data within the spectral sampling region from the ultrasound echo data based on a positional relationship between the spectral sampling region and the multimodal ultrasound image, wherein the extracted ultrasound echo data within the spectral sampling region contains the ultrasound echo data from the plurality of receiving scan lines;
performing spectral analysis on the extracted the ultrasound echo data within the spectral sampling region to obtain a spectral image at the spectral sampling region; and
displaying the spectral image.

16. The method according to claim 1, further comprising:
configuring at least one spectral sampling region at a target blood vessel in the multimodal ultrasound image, such that ultrasound echo data within the spectral sampling region contains ultrasound echo data from a plurality of receiving scan lines;
determining an ultrasound scanning parameter based on the spectral sampling region;
performing a second ultrasound scan on a first region that at least contains the spectral sampling region based on the ultrasound scanning parameter, such that the spectral sampling region contains the plurality of receiving scan lines, and acquiring ultrasound echo data of the first region, wherein the first region is greater than or equal to the spectral sampling region;
extracting the ultrasound echo data within the spectral sampling region from the ultrasound echo data of the first region, wherein the extracted ultrasound echo data within the spectral sampling region contains the ultrasound echo data from the plurality of receiving scan lines;
performing spectral analysis on the extracted ultrasound echo data within the spectral sampling region to obtain a spectral image at the spectral sampling region; and
displaying the spectral image.

17. The method according to claim 1, further comprising:
transmitting ultrasound waves to the region of interest within the target tissue, and acquiring ultrasound echo data based on echoes of said ultrasound waves; or, acquiring ultrasound echo data based on the echoes of the ultrasound waves transmitted during the C-mode scanning;
configuring at least one spectral sampling region at a target blood vessel in the multimodal ultrasound image, such that an angle is formed between a major axis of the at least one spectral sampling region and a beam propagation direction of ultrasound waves transmitted to said spectral sampling region;
extracting ultrasound echo data within the spectral sampling region from the ultrasound echo data based on a positional relationship between the spectral sampling region and the multimodal ultrasound image;
performing spectral analysis on the extracted ultrasound echo data within the spectral sampling region to obtain a spectral image at the spectral sampling region; and
displaying the spectral image.

18. The method according to claim 1, further comprising:
configuring at least one spectral sampling region at a target blood vessel in the multimodal ultrasound image, such that an angle is formed between a major axis of the at least one spectral sampling region and a beam propagation direction of ultrasound waves transmitted to said spectral sampling region;
determining an ultrasound scanning parameter based on the spectral sampling region;
performing a second ultrasound scan on a first region that at least contains the spectral sampling region based on the ultrasound scanning parameter to obtain ultrasound echo data of the first region, wherein the first region is greater than or equal to the spectral sampling region;
extracting ultrasound echo data within the spectral sampling region from the ultrasound echo data of the first region;
performing spectral analysis on the extracted ultrasound echo data within the spectral sampling region to obtain a spectral image at the spectral sampling region; and
displaying the spectral image.

19. The method according to claim 15, wherein the at least one spectral sampling region comprises a closed region and a first straight line that passes through the closed region; wherein:
a size of the closed region matches a size of the target blood vessel;
a shape of the closed region matches a shape of the target blood vessel; and
an extension direction of the first straight line coincides with a blood flow direction within the target blood vessel.

20. An ultrasound imaging device, comprising:
an ultrasound probe;
a transmit circuit, configured to excite the ultrasound probe to transmit ultrasound waves;
a receive circuit, configured to control the ultrasound probe to receive echoes of the ultrasound waves; and
a processor, configured to execute the method according to claim 1.
